(19) United States
(12) Patent Application Publication
(10) Pub. No.: US 2017/0159112 A1
(43) Pub. Date: Jun. 8, 2017

(54) AMPLIFIED ISOTHERMAL DETECTION OF POLYNUCLEOTIDES WITH ATP RELEASE

(71) Applicant: The Board of Trustees of the Leland Stanford Junior University, Stanford, CA (US)

(72) Inventors: Eric T. Kool, Stanford, CA (US); Debin Ji, Palo Alto, CA (US); Michael G. Mohsen, Stanford, CA (US)

(21) Appl. No.: 15/366,898

(22) Filed: Dec. 1, 2016

Related U.S. Application Data
(60) Provisional application No. 62/262,274, filed on Dec. 2, 2015.

(51) Int. Cl. C12Q 1/68; C07H 19/207

(52) U.S. Cl. CPC C12Q 1/6823; C12Q 1/6853; C12Q 1/6827; C07H 19/207

(57) ABSTRACT

The presence of a target polynucleotide sequence of interest, including targets comprising genetic variations or a single nucleotide polymorphism, is detected by a DNA polymerization reaction, where the reaction mixture includes mixtures of nucleotides including at least one chimeric nucleoside tetraphosphate dimer ATP-linked nucleotide (ARN), in which ATP is the leaving group. DNA synthesis with ARNs is shown to be sequence specific, based on priming with a primer or template complementary to a target sequence. The released ATP is assayed in a qualitative or quantitative analysis, where one equivalent of ATP is released for every deoxynucleotide incorporated from an ARN.

### CROSS REFERENCE

This application claims benefit of U.S. Provisional Patent Application No. 62/262,274, filed Dec. 2, 2015, which application IS incorporated herein by reference in its entirety.

### FEDERALLY SPONSORED RESEARCH AND DEVELOPMENT

This invention was made with Government support under contracts GM110050, GM068122 awarded by the National Institutes of Health. The Government has certain rights in the invention.

### BACKGROUND OF THE INVENTION

Methods for detecting polynucleotides are broadly useful in biology and medicine, and the majority of applications use luminescence signals in the detection. For example, fluorescent signals are important for reporting on the presence and quantities of RNA and DNA in real-time PCR; multiple molecular approaches exist for this application, including the use of DNA-binding dyes such as Oregon Green, and fluorogenic probes such as “Taq-Man” probes. Detection of DNA and RNA in cellular specimens is also useful; this is commonly carried out by polymerase incorporation of BrdU with subsequent fluorescent antibody detection, or by incorporation of other functional groups into DNA that can later be detected by bioorthogonally reactive fluorescent dyes.

The use of luciferase signaling is widely applied in biology and medicine, and provides the advantage of very low background signals from the enzymatically triggered chemiluminescence. For example, luciferase is commonly used in ELISA and other “sandwich” assays of proteins. However, it has been rarely used in reporting on DNA. One of the only existing examples is the “pyrosequencing” methodology developed for high-throughput DNA sequencing (see Ronaghi et al. Science 1998, 281, 363-365). In this technology, four enzymes are employed. DNA polymerase copies a template strand, generating pyrophosphate. Two additional enzymes (ATP sulfurylase and apyrase) recycle the pyrophosphate product of the DNA polymerase reaction, generating modified ATP, which can then ultimately be detected via the fourth enzyme, luciferase. This method is highly sensitive, but is also complicated, given the need for several enzymes and a relatively complex reaction mixture. As a result, the method is not used beyond its application in pyrosequencing instruments.

Further improvements in methods for general signaling of DNA and RNA sequences via polymerase synthesis may be useful in amplified detection of native nucleic acids, in reporting on isothermal amplification methods such as rolling circle amplification (RCA), and in future-generation approaches to DNA sequencing. See, for example, Fire and Xu, Proc. Natl. Acad. Sci. USA 1995, 92, 4641-4645; Liu et al. J. Am. Chem. Soc. 1996, 118, 1587-1594; Lizardi et al. Nat. Genet. 1998, 19, 225-232; Yi et al. Nucleic Acids Res. 2006, 34, e81.

To this end, it would be desirable to take advantage of the high sensitivity and specificity of the luciferase enzyme in detecting DNA synthesis, but to avoid the complexity inherent in the four-enzyme pyrosequencing strategy. The present invention provides such methods and compositions.

Publications of interest include U.S. Pat. No. 7,682,809, “Direct ATP release sequencing”. The '809 patent teaches methods in which one ARN is present in a sequencing reaction in the absence of any dNTPs, which reaction chemistry is essential for the purpose of sequencing. In contrast, reactions in which all four nucleotides are present cannot be used for sequencing due to the loss of specific information about the position of a nucleotide in the target.

U.S. Pat. No. 7,560,254, “Allele specific primer extension”; U.S. Pat. No. 7,563,574, “Methods, systems and compositions for monitoring enzyme activity and applications thereof”; U.S. Pat. No. 7,981,604, “Methods and kits for analyzing polynucleotide sequences”; Pojoba et al. (2004) Biochem Biophys Res Commun. 315(3):756-62; and Ogilvie (1981) Anal Biochem. 1981 August; 115(2):302-7.

### SUMMARY OF THE INVENTION

Compositions and methods are provided for the sequence specific detection of polynucleotides, including mRNA, genomic DNA, extrachromosomal DNA, miRNA and other small sequences, rRNA, viral RNA, etc., in a variety of platforms. Samples suitable for analysis include isolated polynucleotides; cell lysates; whole cells and tissues. Kits for practice of the methods are also provided.

In the methods of the invention, the presence of a target polynucleotide sequence of interest is detected by a polymerization reaction, where the reaction mixture includes at least one chimeric nucleoside tetraphosphate dimer in which ATP is the leaving group. Such dimers are referred to as ATP-releasing nucleotides (ARNs). DNA synthesis with ARNs is shown herein to be sequence specific, showing clear nucleotide/template base selectivity, based on priming with a primer or template complementary to the sequence of interest. The presence of an adenosine linkage at the terminus of an ARN does not prevent efficient and selective synthesis with multiple DNA polymerases and reverse transcriptases.

In some embodiments of the invention, methods are provided for detection of specific alleles in a polynucleotide sample, where the allelic variation may include, without limitation, single nucleotide polymorphisms, gene rearrangements, single nucleotide deletions, single nucleotide insertions, etc. Polynucleotide sample include, without limitation, mRNA or other class of RNA, amplified cDNA, genomic DNA, etc. In such methods the presence of an allelic form of a sequence is detected by polymerization reactions, where the reaction mixture includes at least one chimeric nucleoside tetraphosphate dimer in which ATP is the leaving group. Primers are designed to be complementary to one or more of the allelic forms, where the terminal 3′ nucleotide of the primer is designed to be specific to a position of variation. The method exploits the activity profile of polymerase enzymes, which are more efficient at extending primer termini that are correctly matched than termini that are mismatched. The released ATP from a reaction for each of the primers is assayed, where a significantly larger release of ATP is found where there is a perfect match between the primer and the sequence that is present in the polynucleotide sample. A comparison of the ATP release allows determination of which allele is present.

In contrast to sequencing reactions, in which only a single dNTP is present in any given reaction, a reaction mixture of the methods of the present invention comprises a combination of dNTPs and ARNs that is sufficient to provide a substrate for all bases present in the target polynucleotide. Generally all four deoxynucleotides are present in a reaction mix, where each deoxynucleotide is provided either as a native dNTP, or as an ARN, e.g. deoxyadenosine-5′-tetraphosphate-P4-5′-adenosine (dCppppA), deoxycytidine-5′-tetraphosphate-P4-5′-adenosine (dAppppA), deoxyguanosine-5′-tetraphosphate-P4-5′-adenosine (dGppppA) or deoxythymidine-5′-tetraphosphate-P4-5′-adenosine (dTppppA).

In some embodiments, the four deoxynucleotides are provided as two ARNs, and two native dNTPs. In some embodiments, the four deoxynucleotides are provided as three ARNs, and one native dNTP. In some embodiments, all four ARNs are present. For any given base, the reaction mixture will usually contain a native dNTP or an ARN, but not both. Surprisingly, a subset of ARNs combined with dNTPs may provide a stronger signal than a reaction with all four ARNs.

The released ATP can be assayed in a qualitative or quantitative analysis, where one equivalent of ATP is released for every deoxynucleotide incorporated from an ARN. Any convenient method for the detection of ATP can be used, as known in the art, including without limitation: luciferase bioluminescence assays, fluorescent dyes, target-responsive aptasensors, and the like. In some such embodiments, the detection reagent(s) is combined with the reaction mixture after the polymerization reaction is substantially complete, e.g. where a desired level of the product of the reaction has accumulated, such as after at least about 15 minutes, after at least about 30 minutes, after at least about 1 hours, after at least about 2 hours, after at least about 4 hours, after at least about 6 hours, after at least about 12 hours, after at least about 18 hours, after at least about 24 hours or more. In other such embodiments the detection reagent(s) is combined with the reaction mixture at or close to the initiation of the reaction, where the enzymes can be provided as separate entities or as a fusion protein of polymerase and luciferase.

In some embodiments, the methods of the invention assay for ATP by detecting light produced by luciferase in the presence of ATP and luciferin. It is shown herein that while ARNs are efficient substrates for DNA polymerase, they are inefficient with luciferase, thus minimizing background signal.

In some embodiments, a sample comprising, or suspected of comprising, the target polynucleotide is combined a template in the reaction mixture, and wherein the template is a circular DNA having a region complementary to a sequence of interest in the target polynucleotide. Reactions can provide for synthesis by rolling circle or by branched rolling circle amplification. In such embodiments, the target polynucleotide may be a short polynucleotide, e.g. a polynucleotide of less than about 35 nt in length, less than about 30 nt in length, less than about 25 nt. in length, less than about 20 nt. in length, that acts as a primer for a rolling circle reaction. In some such embodiments the target polynucleotide is an miRNA, which are generally from about 20 to about 25 nt. in length.

Methods for the synthesis of ARNs are also provided. In one embodiment, an ARN is synthesized in a one pot reaction, where salts of standard deoxynucleoside monophosphates (dNMPs) are activated and then reacted with a salt of 5′-ATP to produce the desired chimeric dimers, or where a salt of adenosine monophosphate (AMP) is activated and then reacted with salts of different deoxynucleotide-5′-triphosphates (dNTPs).

Applications for methods of the invention include in vitro diagnostics, including clinical diagnostics, research in the fields of molecular biology, high throughput drug screening, veterinary diagnostics, agricultural-genetics testing, environmental testing, food testing, industrial process monitoring, etc. In vitro diagnostics and clinical diagnostics relate to the analysis of nucleic acid samples drawn from the body to detect the existence of a disease or condition, its stage of development and/or severity, and the patient's response to treatment. In high throughput drug screening and development, nucleic acids are used to analyze the response of biological systems upon exposure to libraries of compounds in a high sample number setting to identify drug leads. Veterinary diagnostics and agricultural genetics testing provide a means of quality control for agricultural genetic products and processes. In environmental testing, organisms and their toxins that characterize an environmental medium, e.g. soil, water, air, etc., are analyzed. Food testing includes the qualitative identification and/or quantitation of organisms, e.g. bacteria, fungi, etc., as a means of quality control.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. Structures and strategy in this study. (A) The four chimeric ATP linked deoxynucleotides. (B) Scheme showing how DNA polymerase activity incorporates the deoxynucleotide portion of an ARN while copying a template, releasing ATP, which can subsequently activate luciferase luminescence signaling.

FIG. 2. Initial primer extension studies of chimeric nucleotides with Kf (exo.)

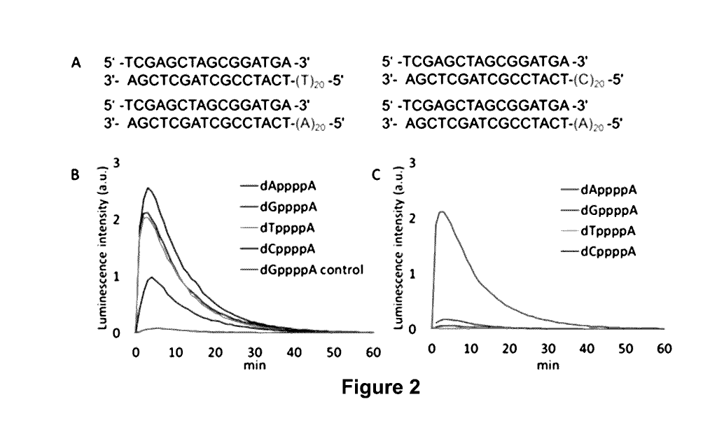

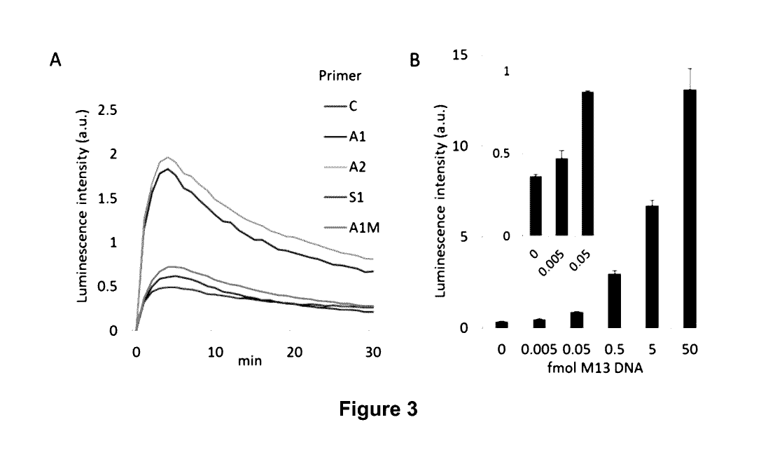

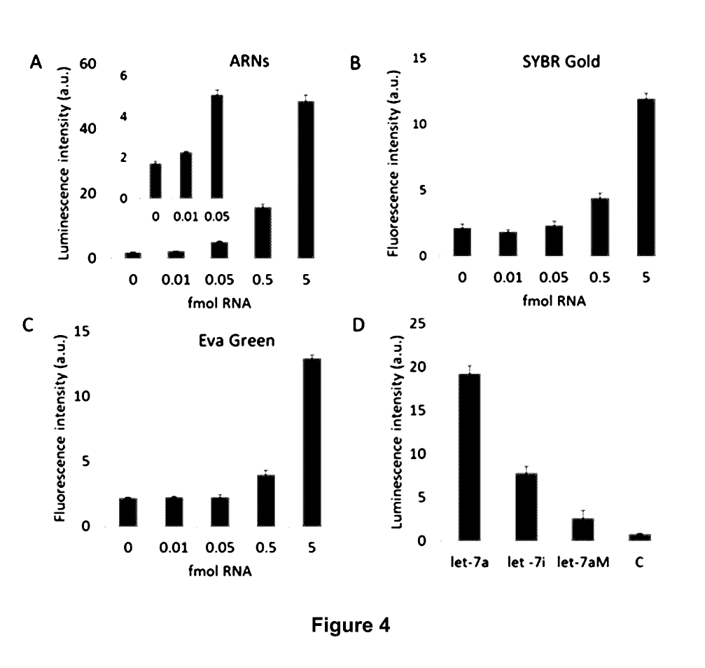

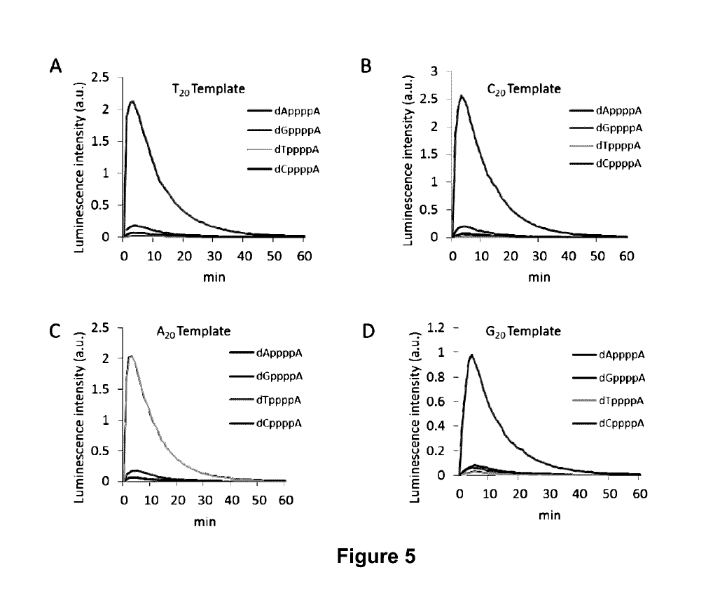

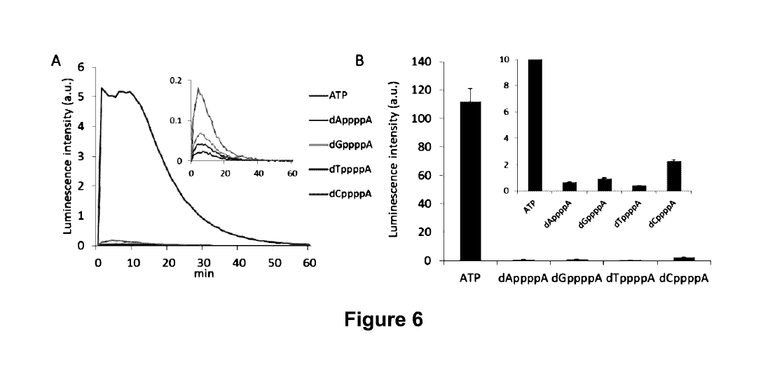

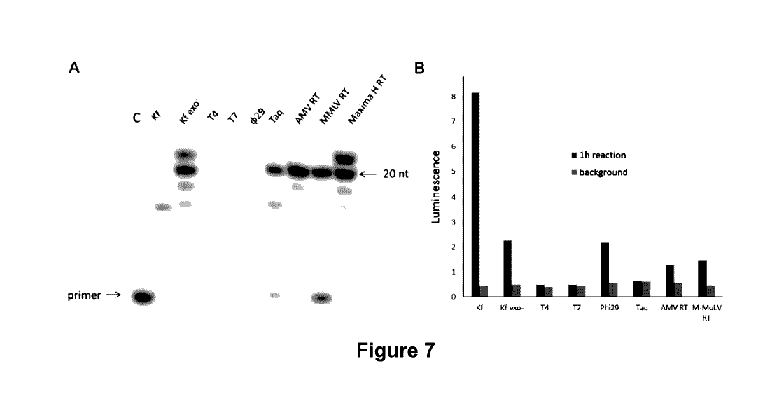

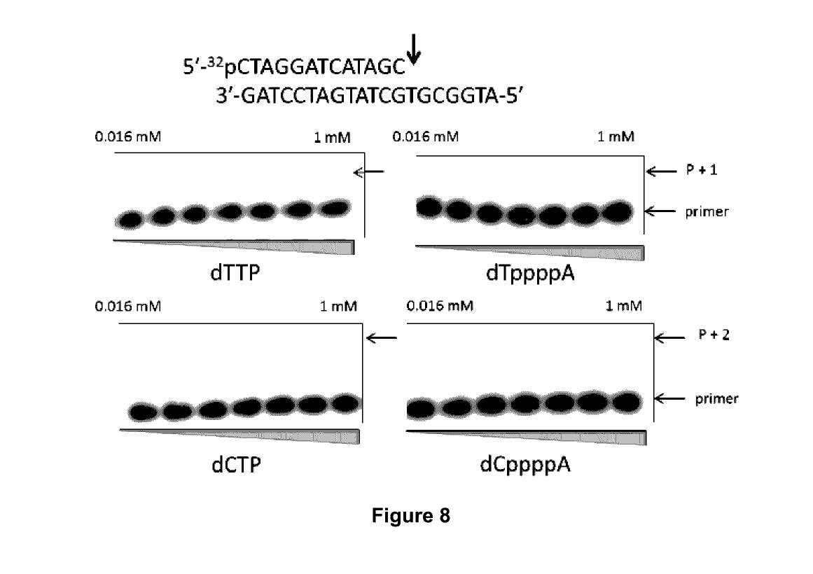

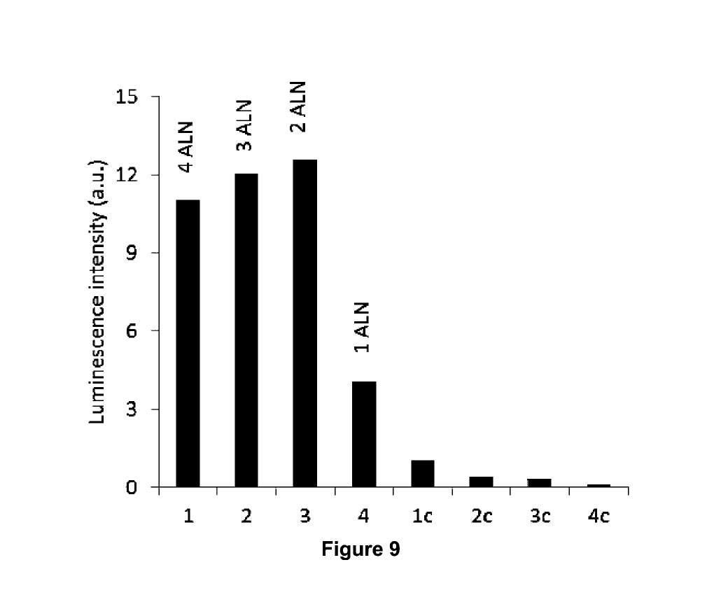

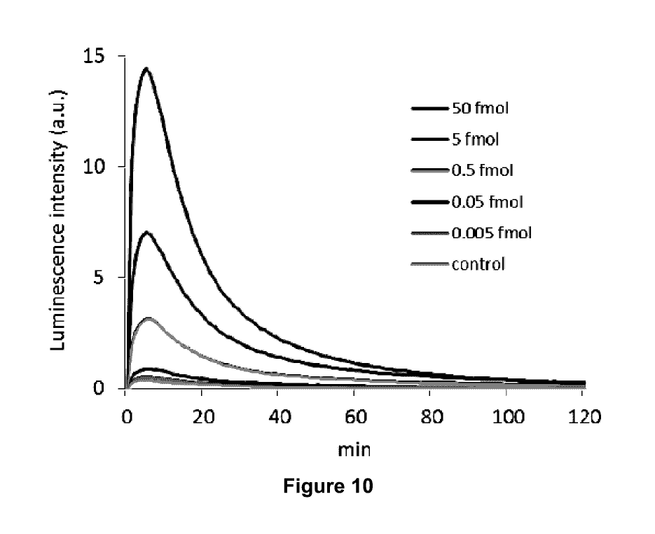

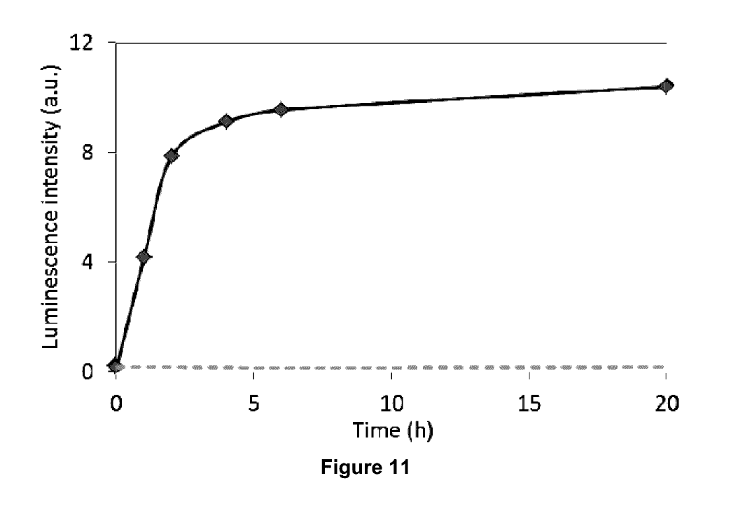

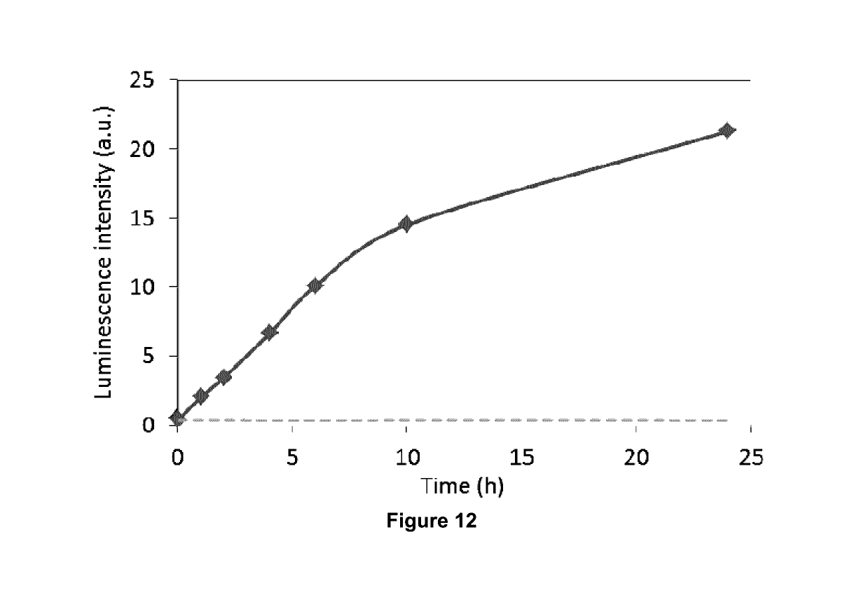

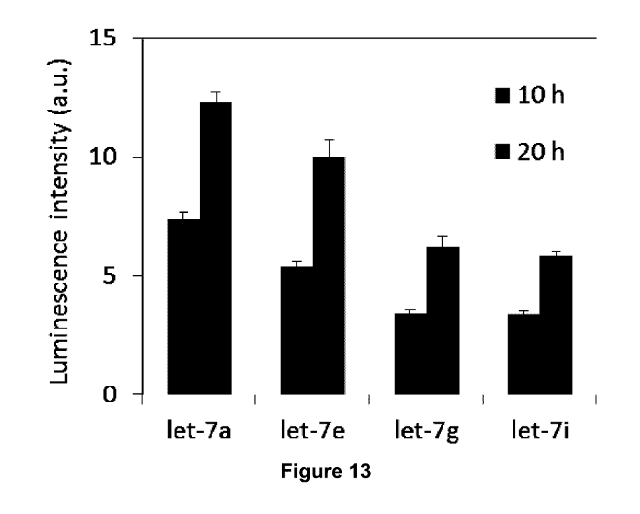

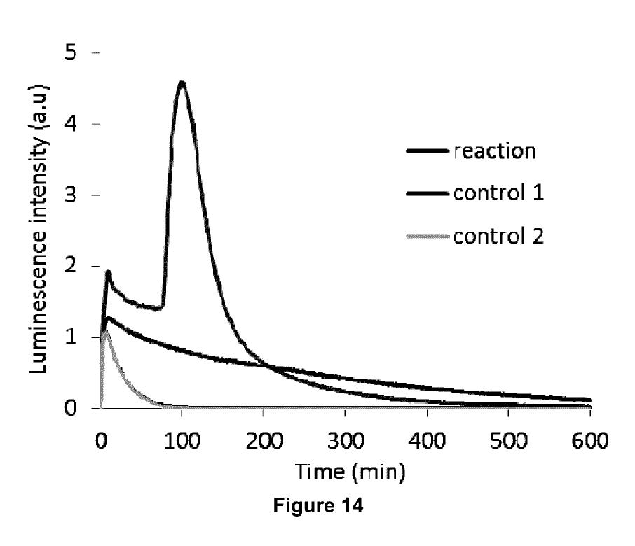

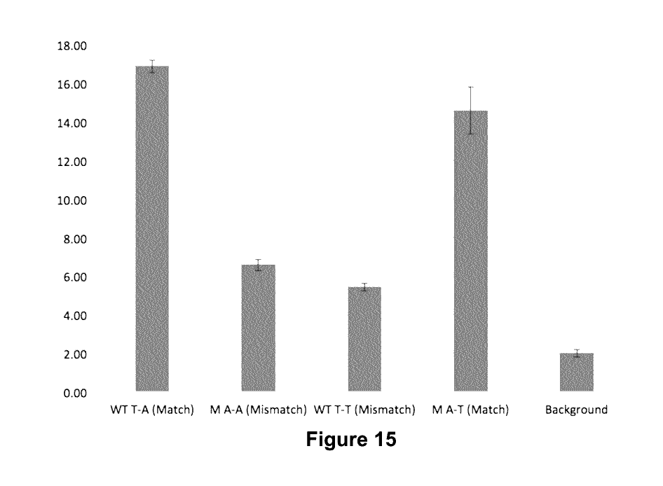

polymerase. (A) Primer-template duplexes with (N)20 ends used in this study. (B) Luminescence signals resulting from the incorporation of ATP-linked nucleotides by Kf (exo.) polymerase. The Kf (exo.) polymerase reaction was carried out with 20 μM chimeric nucleotides and 1 μM corresponding primer template at 37° C. for 1 h. 5 μL of polymerase reaction solution were used for the luciferase reaction. The bioluminescence signal was recorded in 1 min intervals for 1 h. dGppppA control means no primer was added. (C) Kf (exo.) polymerase selectivity with chimeric nucleotides. The reaction was carried out using the (T)₂₀ template and each of the four ARNs under the same reaction conditions as FIG. 2B.

FIG. 3. Detection of circular M13 DNA using chimeric nucleotides and luciferase. (A) Signals with varied primers on M13 DNA. Luminescence signal from 5 μL of polymerase reaction with 1 nM primer and 1 nM M13 DNA at 37° C. for 5 h. A1 and A2 are antisense M13 DNA primers; A1M is the A1 primer mismatched at the three 3′-terminal nucleotides; S1 is a non-complementary sense M13 primer; and “C” is a control with primer A1 but lacking DNA. (B) Testing limit of detection of M13 DNA. Polymerase reactions were carried out with 0.005 to 50 fmol of primer A1/phage DNA at 37° C. for 24 h. Luciferase signals are shown as the 5-minute values; error bars represent standard deviations from three replicates.

FIG. 4. Detection of miRNA with chimeric nucleotides. (A) Measuring limit of detection of miRNA let-7a using chimeric nucleotides. The branched RCA reactions were carried out simultaneously with varied amounts of miRNA let-7a at 30° C. for 24 h. Then 5 μL polymerase reaction and 95 μL luciferase reaction mixtures were combined and the luminescence signals at 5 min were recorded. Error bars represent the standard deviation from three trials. (B) Measuring limit of detection of let-7a RNA using SYBR Gold Dye (emission at 538 nm). (C) Measuring limit of detection of miRNA using EvaGreen Dye (emission at 525 nm). (D) Test of selectivity among related let-7 RNA family members and a mismatched version (let-7aM) (20 h polymerase reaction). Luminescence signals were measured at 5 min.

FIG. 5. Selectivity of chimeric nucleotides with Kf (exo-) polymerase. Each of the four chimeric ARNs was supplied with the polymerase and the annealed primer-template duplex immediately upstream of the sequence T₂₀ (A), C₂₀ (B), A₂₀ (C), and G₂₀ (D), respectively. The polymerase reaction contained: 20 μM chimeric nucleotide, 1 μM corresponding primer/template and 1 μL Kf (exo-) polymerase in manufacturer's polymerase reaction buffer. After 1 h incubation at 37° C., 5 μL reaction solutions were added to 95 μL luciferase reaction solution. The bioluminescence signal was recorded at 1 min intervals over 1 h by microplate fluorimeter.

FIG. 6. Testing dinucleotides as substrates for luciferase. Luminescence readings over 60 min with either 1 μM ATP or 1 μM chimeric nucleotides shown, reacted in 100 μL luciferase reaction solution. (A) Time course of luciferase background signals. (B) Summed signals over 60 min. Error bars show standard deviation over 3 measurements. Insets show the same data with magnified scales.

FIG. 7. Screening varied DNA polymerases and reverse transcriptases with chimeric nucleotides using primer extension experiments on short linear templates. (A) PAGE gel showing primer extension with ARNs after 1 h on 20mer template containing all four bases (sequence below (“Steady state kinetics” section), N=T). C is 13mer radiolabeled primer alone. Standard 20 μL polymerase reactions contained: 0.1 μM annealed primer/template, four chimeric ATP-linked nucleotides 20 μM each, 1× reaction buffer and 0.5 μL polymerase or reverse transcriptase. The reaction mixture was incubated at 37° C. for 1 h (except 4)29 polymerase at 30° C. and Taq DNA polymerase at 65° C.). Note that T4 and T7 DNA polymerases have strong 3′→5′ exonuclease activity, which appears to digest the primer after during extended reaction times. (B) Relative luciferase signals after 1 h reaction with varied polymerases on a primer/template duplex (1 μM) with four ARNs (20 μM). Background data are signals from the same mixture without primer/template DNA. Primer/template is SEQ ID NO:1 5-TCGAGCTAGCGGATGA-3′/SEQ ID NO:2 GAGGAAGGAGGAGGAGGAGGTCATCCGCTAGCTCGA-3′. Luciferase signals were measured with 5 μL reaction solution, analyzing with 95 μL luciferase reaction mixture (3 min time point shown).

FIG. 8. Representative gel images for measuring nucleotide incorporation opposite a template dT. Reactions were conducted in the presence of individual dNTPs (dTTP and dCTP) or chimeric ATP-linked nucleotides (dTppppA or dCppppA) with the concentration range 0.016 mM to 1 mM. The concentration ratios between neighboring lanes were 0.50.

FIG. 9. Screening different combinations of natural dNTPs and chimeric ATP-linked nucleotides for maximizing signal over background. Polymerase reactions carried out with 50 fmol annealed primer/M13 DNA and 20 μM each of the nucleotides at 37° C. for 20 h. 5 μL of this reaction solution was then added to 95 μL luciferase reaction mixtures and the luminescence signals at 5 min were recorded. The different combinations of nucleotides are: 1 (dAppppA, dGppppA, dTppppA and dCppppA), 2 (dAppppA, dGppppA, dTppppA and dCTP), 3 (dAppppA, dGppppA, and dTTP, dCTP), 4 (dAppppA and dGTP, dTTP, dCTP). 1c, 2c, 3c, 4c show data for the corresponding control reactions without polymerase.

FIG. 10. Luminescence measurement of the limit detection of M13 DNA. Polymerase reactions were carried out with varied concentration of DNA/primer A1 at 37° C. for 24 h. Reactions conditions were as in Fig. S5 legend except with varied DNA concentration. After polymerase reaction, 5 μL was added to 95 μL luciferase reaction mixture and the luminescence signals were recorded at 1 min intervals.

FIG. 11. Time course of signal in the detection of M13 DNA by phi29 polymerase. Polymerase reactions conditions were as in Fig. S5 legend with 50 fmol annealed primer/M13 DNA. After polymerase reaction for the times shown, 5 μL was added to 95 μL luciferase reaction mixture and the luminescence signals were recorded at 5 min. Dashed line indicates level of background signal with no M13 DNA.

FIG. 12. Time course of signal in the polymerase reaction of let-7a miRNA. Polymerase reactions carried out with 5 fmol let-7a miRNA and 10 nM small circular ODN, 50 μM each of chimeric ATP-linked nucleotides (dAppppA and dGppppA) and 50 μM natural nucleotides (dTTP and dCTP), 1 μM primer stock (SEQ ID NO:3 5′-TCTCTCGTGCAGACT-3′), 1× polymerase reaction buffer and 1 μL 4)29 DNA polymerase. Reactions were run for the times shown. 5 μL of this reaction solution was then added to 95 μL luciferase reaction mixtures and the luminescence signals at 5 min were recorded. Dashed line indicates level of background signal with no miRNA.

FIG. 13. Test of selectivity among closely related let-7 family members. See main text FIG. 4D for RNA targets; further experiments were carried out here with let-7 DNA variants. Polymerase reactions were carried out with 1 nM let-7 family members at 37° C. for 10 h or 20 h. Reactions conditions were as in “detection of let-7a miRNA with branched RCA”. Luminescence signals were measured at 5 min.

FIG. 14. Single-tube polymerase and luciferase reactions with phage M13 DNA. 10 nM annealed M13mp18 single-stranded DNA/primer complex, 20 μM each of chimeric ATP-linked nucleotides (dAppppA and dGppppA) and 20 μM natural nucleotides (dTTP and dCTP), and 2 μL Kf polymerase were mixed with 100 μL luciferase reaction buffer. Control 1 was the reaction without primer/M13; control 2 was the reaction without primer/M13 and Kf polymerase.

FIG. 15. Luciferase signals from experiments detecting and identifying BRAF single nucleotide variations in RNA. VVT RNA is present in first two lanes; MUT RNA in lanes 3,4. Conditions: BRAF 520mer RNA targets: 100 nM target, 1 μM allele-specific primer, 20 μM ARNs, Maxima H Minus RT, 30 min at 37° C.

### DETAILED DESCRIPTION OF THE INVENTION

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing those components that are described in the publications that might be used in connection with the presently described invention.

As used herein, compounds which are “commercially available” may be obtained from standard commercial sources including Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hannover, Germany), Spectrum Quality Product, Inc. (New Brunswick, N.J.), TCI America (Portland Oreg.), Trans World Chemicals, Inc. (Rockville Md.), Wako Chemicals USA, Inc. (Richmond Va.); Molecular Probes (Eugene, Oreg.); Applied Biosystems, Inc. (Foster City, Calif.); and Glen Research (Sterling, Va.).

As used herein, “suitable conditions” for carrying out a synthetic step are explicitly provided herein or may be discerned by reference to publications directed to methods used in synthetic organic chemistry. The reference books and treatise set forth above that detail the synthesis of reactants useful in the preparation of compounds of the present invention, will also provide suitable conditions for carrying out a synthetic step according to the present invention.

As used herein, “methods known to one of ordinary skill in the art” may be identified through various reference books and databases. Suitable reference books and treatise that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

ATP-releasing nucleotides (ARNs). As used herein, the term ARN refers to a chimeric DNA nucleoside tetraphosphate dimer comprising ATP. Use of one or more ARN as a substrate for a template dependent polymerization reaction results in the incorporation of the dNMP substituent into the elongating primer or template, and the corresponding release of the ATP substituent. As shown with reference to formula (I), the subject ARN compounds contain an adenosine substituent linked via four phosphate groups to a 2′-deoxynucleoside substituent. ARNs have the general structure:

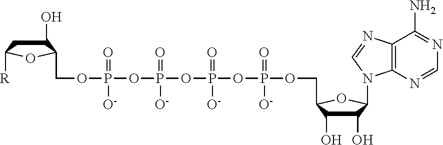

where R is any purine or pyrimidine including substituted purines or pyrimidines. R groups of interest include adenine (A), thymine (T), guanine (G), cytosine (C), or an analog thereof, where an analog has a modified base retains an ability to base pair with a complementary nucleotide. These ARNs may also be referred to individually as, for example, deoxyadenosine-5′-tetraphosphate-P4-5′-adenosine (dCppppA), deoxycytidine-5′-tetraphosphate-P4-5′-adenosine (dAppppA), deoxyguanosine-5′-tetraphosphate-P4-5′-adenosine (dGppppA) or deoxythymidine-5′-tetraphosphate-P4-5′-adenosine (dTppppA). While drawn as phosphate anions, it is understood that they may be protonated at lower pH values.

The terms “nucleoside”, “nucleotide”, “deoxynucleoside”, and “deoxynucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the “nucleoside”, “nucleotide”, “deoxynucleoside”, and “deoxynucleotide” include those moieties that contain not only conventional ribose and deoxyribose sugars, but other sugars as well.

Nucleotides useful in the invention include naturally occurring, or native, nucleotides and nucleotide analogs. Exemplary nucleotides include phosphate esters of deoxyadenosine, deoxycytidine, deoxyguanosine, deoxythymidine, deoxyuridine, adenosine, cytidine, guanosine, and uridine. Other nucleotides comprise an adenine, cytosine, guanine, thymine base, a xanthine or hypoxanthine; 5-bromouracil, 2-aminopurine, deoxyinosine, or methylated cytosine, such as 5-methylcytosine, and N4-methoxydeoxycytosine. Deoxynucleotide analogues useful in the invention include, without limitation, -5 alkyl, alkenyl, alkynyl, and F, Cl, Br, I pyrimidines, and the same substituents at C7 of 7-deazapurines; 5-methyl C, 5-hydroxymethyl C.

The term a “native dNTP” refers to naturally occurring deoxyribose nucleotide triphosphosphates, as known in the art, e.g. dTTP, dATP, dCTP, dGTP.

“Modified nucleotides”, “modified nucleosides”, “nucleotide analogs”, or “nucleoside analogs” (excluding A, T, G, and C) include for example, nucleotides or nucleosides having a structure derived from purine or pyrimidine (i.e., nucleotide or nucleoside analogs). For example and without limitation, a modified adenine may have a structure including a purine with a nitrogen atom covalently bonded to C6 of the purine ring as numbered by conventional nomenclature known in the art. In addition, it is recognized that modifications to the purine ring and/or the C6 nitrogen may also be included in a modified adenine. A modified thymine may have a structure comprising at least a pyrimidine, an oxygen atom covalently bonded to the C4 carbon, and a C5 methyl group. Again, it is recognized by those skilled in the art that modifications to the pyrimidine ring, the C4 oxygen and/or the C5 methyl group may also be included in a modified adenine. For example and without limitation, a modified guanine may have a structure comprising at least a purine, and an oxygen atom covalently bonded to the C6 carbon. A modified cytosine may have a structure including a pyrimidine and a nitrogen atom covalently bonded to the C4 carbon. Modifications to the purine ring and/or the C6 oxygen atom may also be included in modified guanine nucleotides or nucleosides. Other known modifications to purines include 7-deaza derivatives, such as 7-deazaadenine and 7-deazaguanine. Modifications to the pyrimidine ring and/or the C4 nitrogen atom may also be included in modified cytosine nucleotides or nucleosides.

Analogs may also be derivatives of purines without restrictions to atoms covalently bonded to the C6 carbon. These analogs would be defined as purine derivatives. Analogs may also be derivatives of pyrimidines without restrictions to atoms covalently bonded to the C4 carbon. These analogs would be defined as pyrimidine derivatives. Purine analogs include those having the capability of forming stable base pairs with pyrimidine analogs without limitation to analogs of A, T, G, and C as defined. Purine analogs also include those not having the capability of forming stable base pairs with pyrimidine analogs without limitation to analogs of A, T, G, and C.

The ARN compounds may be made by the methods disclosed herein, for example where salts of deoxynucleoside monophosphates (dNMPs) are activated and then reacted with a salt of 5′-ATP. Alternatively the ARN compounds are made by a method where a salt of adenosine monophosphate (AMP) is activated and then reacted with the salts of different deoxynucleotide-5′-triphosphates (dNTPs).

Salts of deoxynucleoside monophosphates or of ATP that are useful for the methods include, without limitation, tetra- or tri-alkylammonium salts, ammonium, lower alkylammonium, pyridinium, lutidinium, cyclohexylammonium, a metal salt cation such as Na⁺, K⁺, Li⁺, Ba⁺, Mg⁺, or the like as known in the art.

Activating agents include, without limitation carbonyldiimidazole, or a carbodiimide activating agent.

For example, the tetrabutylammonium salt of ATP can be added to carbonyldiimidazole, quenched, and redissolved in anhydrous DMF. The desired deoxynucleoside monophosphate tetrabutylammonium or tributylammonium and anhydrous MgCl₂ are added and the product precipitated and washed with acetone. The desired product can be purified by methods known in the art.

Alternatively the tetrabutylammonium salt of a dNTP is added to carbonyldiimidazole, quenched, and redissolved in anhydrous DMF. The adenosine monophosphate tetrabutylammonium or tributylammonium and anhydrous MgCl₂ are added and the product precipitated and washed with acetone. The desired product can be purified by methods known in the art. Although in principle both of these synthetic methods produce the same desired compounds, this latter approach has the surprising benefit that the reactions and compounds have never been exposed to ATP, and so have little or no ATP present as a minor contaminant in the ARN product. This is important because it lowers the amount of background signal.

The ARNs can also be produced by enzymatic methods e.g., using a pyrophosphohydrolase, such as the E. coli pyrophosphohydrolase, as described in Plateau, P., et al., (1985) Biochemistry 24, 914-922). Alternative synthetic methods include, without limitation, phosphitylation of a protected nucleoside with 2-chloro-4H-1,3,2-benzo-dioxaphosphorin-4-one (salicylchlorophosphite), followed by sequential reaction with inorganic pyrophosphate and a nucleoside 5′-monophosphate.

A “target sequence” or “sequence of interest” refers to the particular nucleotide sequence of the target polynucleotide that can be hybridized to a primer or complementary template. Exemplary targets include any DNA or RNA sequence, e.g. viral polynucleotides, bacterial polynucleotides, and eukaryotic polynucleotides, where the target sequence can be rRNA, mRNA, miRNA, cell-free DNA, genomic DNA, mitochondrial DNA, etc. While the target polynucleotide may be single stranded or double-stranded in its native state, typically it will be denatured prior to contacting with a primer or template.

The term “nucleic acid” and “polynucleotide” are used interchangeably herein to describe a polymer of any length, e.g., greater than about 10 bases, greater than about 100 bases, greater than about 500 bases, greater than 1000 bases, usually up to about 10,000 or more bases composed of nucleotides, e.g., deoxyribonucleotides or ribonucleotides, or compounds produced synthetically that can hybridize with naturally occurring nucleic acids in a sequence specific manner analogous to that of two naturally occurring nucleic acids, e.g., can participate in Watson-Crick base pairing interactions.

As used herein, a “test sample” is a sample suspected of containing nucleic acids to be analyzed for the presence or amount of the target polynucleotide. Nucleic acids of the test sample may be of any biological origin, including any tissue or polynucleotide-containing material obtained from a human. For example, the nucleic acids of the test sample may be from a biological sample that may include one or more of: tissue or organ lavage, sputum, peripheral blood, plasma, serum, bone marrow, biopsy tissue including lymph nodes, respiratory tissue or exudates, gastrointestinal tissue, cervical swab samples, semen or other body fluids, tissues or materials. Biological samples may be treated to disrupt tissue or cell structure, thereby releasing intracellular components into a solution which may contain enzymes, buffers, salts, detergents and the like. Alternative sources of nucleic acids may include water or food samples that are to be tested for the presence of a particular analyte polynucleotide that would indicate the presence of a microorganism.

A test sample may comprise DNA, RNA, etc., including total mixed RNA from a biological sample, purified RNA subsets such as mRNA, rRNA etc. In cases where insufficient quantities of RNA can be obtained from the sample, PCR or other known amplification methodology can be used to amplify the sequence of interest prior to analysis. A PCR amplicon can be used to generate complementary RNA, which can be analyzed by the methods of the invention. Alternatively, a PCR amplicon can be analyzed directly, either by separating strands to allow primer access, or by denaturing the amplicon to allow primer access.

The term “primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. A primer serves as an initiation point for nucleotide polymerization catalyzed by either DNA polymerase, RNA polymerase or reverse transcriptase. In the methods of the invention, a primer is usually complementary to a target sequence. For distinction between alleles, two or more primers, each of which is complementary to an allelic sequence can be used in the methods. In some embodiments the allele specific primer is designed such that the terminal 3′ nucleotide of the primer is positioned opposite a position of variation.

Primers are usually of a sufficient length to specifically hybridize to, and initiate synthesis from, the target polynucleotide. A primer can be, for example, of at least about 6 bases in length, more usually at least 7, 8, or 9 bases; for many embodiments of the invention, oligonucleotides are at least 10 bases, at least 12 bases, at least about 14 bases, at least about 16 bases, and not more than about 50 bases in length, usually not more than about 30 bases in length, not more than 25 bases in length, or any length range between any two of these lengths.

As is known in the art, a primer may further comprise a non-complementary region, e.g. to provide for indexing, bar-coding, tags, and the like.

Primers may comprise native nucleic acids, e.g. DNA or RNA, or may comprise modified nucleotides, for example to enhance stability of hybridization. Modified nucleic acids of interest include, without limitation, locked nucleic acid (LNA), 2′-O-methyl RNA, etc.

The term “template” denotes a nucleic acid molecule that can be used by a nucleic acid polymerase to direct the synthesis of a nucleic acid molecule that is complementary to the template according to the rules of Watson-Crick base pairing. For example, DNA polymerases utilized DNA to synthesize another DNA molecule having a sequence complementary to a strand of the template DNA. RNA polymerases utilize DNA as a template to direct the synthesis of RNA having a sequence complementary to a strand of the DNA template. DNA reverse transcriptases utilize RNA to direct the synthesis of DNA having a sequence complementary to a strand of the RNA template.

In specific methods of the invention, a template can contain a portion of sequence that is complementary to the target sequence, in particular where the target sequence is a short polynucleotide, e.g. a polynucleotide of less than about 35 nt in length, less than about 30 nt in length, less than about 25 nt. in length, less than about 20 nt. in length. The remaining portion of the template need not be complementary to the target sequence. A template can be a circular polynucleotide, that acts as a primer for a rolling circle or a branched rolling circle reaction. Circular templates can be, for example, up to 50 nt. in length, up to 75 nt., up to 100 nt., up to 200 nt., up to 300 nt., up to 400 nt., up to 500 nt., or more.

In methods for detection of an allelic variant, the template may extend beyond the primer for at least about 25 nt., at least about 50 nt., at least about 100 nt., at least about 250 nt., at least about 500 nt., or more, which length provides the amplification signal for distinction between allelic forms.

With respect to the region of complementarity between a primer or template and a target, the sequence may or may not be completely complementary. If not completely complementary, the target and primer or template are at least substantially complementary, such that the amount of mismatches allow specific priming of DNA synthesis. The region of complementarity is usually at least about 6 bases in length, more usually at least 7, 8, or 9 bases; for many embodiments of the invention, at least 10 bases, at least 12 bases, at least about 14 bases, at least about 16 bases, and not more than about 50 bases in length, usually not more than about 30 bases in length, not more than 25 bases in length, or any length range between any two of these lengths. Over the region of complementarity the number of mismatches will usually not be more than about 15% of the total number, not more than about 10%, of the total, not more than about 5% of the total. In other words, the region of complementarity will be at least about 85% identical to the target sequence, at least about 90% identical, at least about 95% identical, and may be 100% identical.

The sequence of the primer or template is selected to be complementary, competitive, mismatched, etc. with respect to a target sequence, as dictated by the specific interests of the method. In some embodiments, probe sequences are chosen to be sufficiently selective that there is a detectable difference between binding to a perfect match at the target, and to a single nucleotide mismatch at the target, e.g. where the 3′ terminal nucleotide of the primer corresponds to the position of variation. A highly selective probe binds with high preference to the exact complementary sequence on a target strand as compared to a sequence that has one or more mismatched bases. Less selective probes are also of interest for some embodiments, where hybridization is sufficient for detectable reactions to occur in the presence of one, two three or more mismatches, where a mismatch may include substitutions, deletions, additions, etc.

The phrase “primer extension conditions” denotes conditions that permit for polymerase mediated primer extension by addition of nucleotides to the end of the primer molecule using the template strand as a template.

The term “complementary, “complement,” or “complementary nucleic acid sequence” refers to the nucleic acid strand that is related to the base sequence in another nucleic acid strand by the Watson-Crick base-pairing rules. In general, two sequences are complementary when the sequence of one can hybridize to the sequence of the other in an anti-parallel sense wherein the 3′-end of each sequence hybridizes to the 5′-end of the other sequence and each A, T, G, and C of one sequence is then aligned with a T, A, C, and G, respectively, of the other sequence.

The term “duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. “Perfectly matched” in reference to a duplex means that the poly- or oligonucleotide strands making up the duplex form a double stranded structure with one another such that every nucleotide in each strand undergoes Watson-Crick base pairing with a nucleotide in the other strand. The term “duplex” may include the pairing of nucleoside analogs, such as deoxyinosine, nucleosides with 2-aminopurine bases, and the like, that may be employed. A “mismatch” in a duplex between two oligonucleotides or polynucleotides means that a pair of nucleotides in the duplex fails to undergo Watson-Crick bonding.

The terms “hybridization”, and “hybridizing”, in the context of nucleotide sequences are used interchangeably herein. The ability of two nucleotide sequences to hybridize with each other is based on the degree of complementarity of the two nucleotide sequences, which in turn is based on the fraction of matched complementary nucleotide pairs. The more nucleotides in a given sequence that are complementary to another sequence, the more stringent the conditions can be for hybridization and the more specific will be the hybridization of the two sequences. Increased stringency can be achieved by elevating the temperature, increasing the ratio of co-solvents, lowering the salt concentration, and the like.

Single nucleotide polymorphisms, frequently called SNPs (pronounced “snips”), are the most common type of genetic variation among people. Each SNP represents a difference in a single nucleotide. SNPs occur once in every 300 nucleotides on average in the human genome, and can be present in expressed sequences, or in non-expressed genomic DNA. The sequence of genomic sequences, including the sequence of the human genome, can be accessed for designing allele specific probes that distinguish between forms of an SNP.

The NCBI Short Genetic Variations database, commonly known as dbSNP, catalogs short variations in nucleotide sequences from a wide range of organisms. These variations include single nucleotide variations, short nucleotide insertions and deletions, short tandem repeats and microsatellites. Short Genetic Variations may be common, thus representing true polymorphisms, or they may be rare. Some rare human entries have additional information associated with them, including disease associations, genotype information and allele origin, as some variations are somatic rather than germline events. An example of a somatic variation is a mutation that leads to cancer. Short nucleotide variation data can be accessed via the SNP homepage. A large number of clinically relevant SNPs are known and published in the art, including those disclosed in the Examples, BRAF, JAK2 kinase, ABL1, etc. One of skill in the art can readily access public information to design a suitable primer set for detecting which allele or combination of alleles is present in a sample of interest.

ATP detection reagent(s). Many reagents and assays are known in the art for use in detecting the presence of ATP. For the purposes of the present invention, these reagents are used to detect ATP released during DNA synthesis, and thus provide a qualitative or quantitative assessment for the presence of the target polynucleotide sequence. ATP detection reagents include without limitation luciferase bioluminescence assays (see, for example, J Appl Biochem 3, 473 (1981); Fraga (2008) Photochemical & Photobiological Sciences 7(2):146-158; Bell et al. (2007) Methods Cell Biol. 80:341-352), fluorescent dyes, target-responsive aptasensors, glass bead microarray, GO-nS nanocomplex platform, and the like. In certain embodiments the assay utilizes detection of light produced by luciferin and luciferase.

Exemplary fluorescent dyes are described, for example in Jose et al. (2007) Org. Lett. 9:1979-1982; Lee et al. (2004) Angew. Chem. Int. Ed. 43:4777-4780; Sancenon et al. (2001) Angew. Chem. Int. Ed. 40:2640-2643; Mizukami et al. (2002) JACS 124:3920-3925; Schneider et al. (2000) JACS 122:542-543; Ojida et al. (2006) Angew. Chem. Int. Ed. 45:5518-5521; Li et al. (2005) Angew. Chem. Int. Ed. 44:6371-6374, each of which is herein specifically incorporated by reference.

Target responsive aptamers are described, for example, by Li & Ho (2008) JACS 130:2380-2381; Li & Lu (2006) Angew. Chem. Int. Ed. 45:90-94; Zayats (2006) JACS 128:13666-13667. Glass bead microarrays are described by McClesky et al. (2003) JACS 125:1114-1115. A GO-nS nanocomplex platform is described by Wang et al. (2013) Anal. Chem. 85:6775-6782. Each of these references is herein specifically incorporated by reference.

The term “luciferase” refers to an adenosine triphosphate (ATP) hydrolase that catalyzes the hydrolysis of ATP into constituent adenosine monophosphate (AMP) and pyrophosphate (PPi) along with the release of light. A luciferase has an activity described as EC 1.13.12.7, according to IUBMB enzyme nomenclature. A luciferase of interest is Photinus luciferin 4-monooxygenase (ATP-hydrolyzing).

Luciferin is a common bioluminescent reporter used for in vitro assays in combination with luciferase. This water soluble substrate for the Firefly luciferase enzyme (e.g. Photinus pyralis, Cypridina, Gaussia, Renilla, etc.) utilizes ATP and Mg²⁺ as co-factors to emit a characteristic yellow-green emission in the presence of oxygen. Many reagents and kits are commercially available for this purpose. When luciferin and luciferase are combined in a reaction mixture comprising ATP, there is an immediate flash of light that reaches peak intensity within 0.3-0.5 seconds. The light then begins to decay rapidly with a half-life around 0.5-1.0 min. The optional addition of Coenzyme A to the reaction mixture prevents the fast reaction decay, extending the half-life of the reaction from 2-5 minutes. Variations of luciferin are also known that yield slower signal generation for convenience.

The term “reagent mix”, as used herein, refers to a combination of reagents, that are interspersed and not in any particular order. A reagent mix is heterogeneous and not spatially separable into its different constituents. Examples of mixtures of elements include a number of different elements that are dissolved in the same aqueous solution, or a number of different elements attached to a solid support at random or in no particular order in which the different elements are not spatially distinct.

The compounds of the invention may contain one or more asymmetric centers and may thus give rise to enantiomers, diastereomers, and other stereoisomeric forms that may be defined, in terms of absolute stereochemistry, as (R)- or (S)- or, as (D)- or (L)-for amino acids. The present invention is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

### Compositions

In some embodiments of the invention, a reaction mixture, or certain components thereof, is provided, which mixture comprises the components required for detecting the presence of a target polynucleotide sequence of interest by a polymerization reaction, where the reaction mixture includes at least one chimeric nucleoside tetraphosphate dimer in which ATP is the leaving group.

A reaction mixture or components thereof, for the present invention comprises a combination of dNTPs and ARNs that is sufficient to provide a substrate for all bases present in the target sequence. Generally all four deoxynucleotides are present in a reaction mix, where each deoxynucleotide is provided either as a native dNTP, or as an ARN. In some embodiments, the four deoxynucleotides are provided as two ARNs, and two native dNTPs. In some embodiments, the four deoxynucleotides are provided as three ARNs, and one native dNTP. In some embodiments, all four ARNs are present. For any given base, the reaction mixture will usually contain a native dNTP or an ARN, but not both.

In some embodiments, two ARNs are provided with 2 dNTPs. While any combination can be used, e.g. by optimizing with a polymerase of interest, in some embodiments a preferred combination includes dAppppA, dGppppA, dTTP and dCTP. The ARN and dNTP in a reaction mixture are typically provided at a working concentration, which may be empirically determined, for example at a concentration of from about 0.1 μM for each dNTP or ARN, at least about 1 μM, at least about 10 μM, at least about 20-25 μM, at least about 35 μM, at least about 50 μM, up to about 75 μM, up to about 100 μM, up to about 250 μM, up to about 500 μM, or more.

In some embodiments the reagent ARN and dNTPs can be provided in a concentrated form, suitable for dilution into a reaction mixture, where the ARN and dNTP reagents may be pre-mixed or separately formulated.

A reaction mixture will also comprise a polymerase at an appropriate concentration to perform the synthetic reaction, e.g. using commercially available enzymes according to the manufacturer's instructions. The Michaelis-Menten constant (Kₘ) of an ARN for the polymerase may be comparable to the Kₘ of natural dNTPs, for example less than about 20 μM. It is shown herein that the kcat values for ARNs are within about 2-fold, within about 1.5-fold of those of native nucleotides, and may useful be used with enzymes where the values are within about 20-fold those of native dNTPs. A number of polymerases have been tested and found to be useful in the methods, including without limitation the commonly used enzymes DNA polymerase I, DNA polymerase I Klenow fragment, DNA polymerase I Klenow fragment 3′-exonuclease deficient variant, Taq polymerase, etc., and a number of reverse transcriptase enzymes, including without limitation AMV reverse transcriptase, MMLV reverse transcriptase, maxima reverse transcriptase, maxima H— reverse transcriptase, etc. In some embodiments a suitable enzyme can be provided in a kit, with the nucleotide reagents.

DNA polymerases useful in the invention may also include, but are not limited to: Pyrococcus furiosus (Pfu) DNA polymerase, Pyrococcus woesei (Pwo) DNA polymerase, Thermus thermophilus (Tth) DNA polymerase, Bacillus stearothermophilus DNA polymerase, Thermococcus litoralis (Tli) DNA polymerase, Stoffel fragment, ThermoSequenase™ (Amersham Pharmacia Biotech UK), Therminator™ (New England Biolabs), Thermotoga maritima (Tma) DNA polymerase, Thermus aquaticus (Taq) DNA polymerase, Pyrococcus kodakaraensis KOD DNA polymerase, JDF-3 DNA polymerase, Deep Vent™ DNA polymerase (New England Biolabs), UlTma DNA polymerase (PE Applied Biosystems), Tgo DNA polymerase (Roche Molecular Biochemicals), E. coli DNA polymerase I, archaeal DP1I/DP2 DNA polymerase II, etc. A polymerase may be subjected to so-called “directed evolution” methods that select for a polymerase with altered affinity for ARN. A variety of such directed evolution methods are known in the art, including but not limited to DNA shuffling, exon shuffling, family shuffling, STEP and random priming of in vitro recombination, exonuclease mediated gene assembly, Gene Site Saturation Mutagenesis, Gene Reassembly, SCRATCHY, DNA fragmentation methods, single-stranded DNA shuffling, and the like.

A reaction mixture will comprise the sample suspected of comprising the target sequence. The polynucleotides present in the sample are denatured according to methods known in the art prior to contacting with the polymerase. Any sample can be analyzed, including without limitation biological samples from an individual or population, food samples, swabs of potentially contaminated surfaces, environmental samples, drug testing samples and the like. As shown here, detection of a target sequence can be accomplished with as little as 1 μM quantities, for example at about 1 pM, 5 pM, 10 pM, 100 pM, 250 pM, 500 pM, 1 nM, 5 nM, 10 nM or more.

The reaction mixture will comprise a template or primer to initiate polymerization, where the template or primer comprises a sequence complementary to the target sequence. Parameters for primer or template are as defined herein. The concentration of template or primer is determined by the specific requirements of the analysis, but is usually at least about 1 pM, at least about 0.5 nM, at least about 1 nM, and may be from about 1 nM to about 100 μM, from about 1 nM to about 10 μM, from about 1 nM to about 1 μM, using guidelines known in the art for the polymerase and similar reaction conditions.

In certain embodiments, including but not limited to embodiments where the target polynucleotide is, for example, less than about 100 nt. in length, less than about 50 nt. in length, less than about 25 nt in length, the target polynucleotide serves as a primer, and a template is added to the reaction mix, where the template comprises a region of complementarity to the target sequence. Such template can be circular, to provide for rolling circular amplification. Templates can also provide for branched rolling circle amplification.

In other embodiments, including but not limited to embodiments where the target polynucleotide is longer than about 20 nt. in length, a primer is included in the reaction mixture, where the primer provides specificity for initiation of synthesis from the target polynucleotide.

The reaction mixture also comprises buffers, salts, etc. as known in the art and appropriate for the polymerase or reverse transcriptase. Inhibitors of nucleases, etc. can also be added. The temperature of the reaction is generally between about 20° C. and 40° C. The pH of the reaction is generally between pH 6 and pH 9. These ranges may be extended.

When changing the concentration of a particular component of the reaction medium, that of another component may be changed accordingly. For example, the concentrations of several components such as nucleotides templates or primers may be simultaneously controlled in accordance with the change in those of other components. Also, the concentration levels of components in the reactor may be varied over time.

The reactions may be multiplexed to perform a plurality of simultaneous syntheses, utilizing such reaction vessels as 96 well plates, etc., as are known in the art.

The ATP produced by the polymerase reaction can be detected in any of a variety of different ways. The released ATP can be accumulated in the reaction mixture and then detected by the addition of ATP-dependent detection reagent(s). In such embodiments the detection reagents are added to the reaction mixture after a period of time and under such conditions that the polymerization reaction has proceeded to a desired degree, e.g. to exhaustion of the substrate or primer, or to an intermediate stage pre-determined for the assay. Usually in such embodiments a reaction proceeds for at least about 15 minutes, at least about 30 minutes, at least about 1 hour, at least about 2 hours, at least about 4 hours, at least about 6 hours, at least about 8 hours, at least about 12 hours, at least about 18 hours, at least about 24 hours or more. Included are, for example, a reaction of about 30 minutes to 24 hours, from about 1 hour to about 12 hours, etc.

In other embodiments, the ATP detection reagent, e.g. a fluorescent dye, chemiluminescent system, aptamer, etc. is included in the reaction mixture at initiation, and measurement of the signal is detected during the polymerization reaction. Regardless of when the detection reagent is included, the concentration, buffer, conditions, etc. are chosen to be appropriate for the reagent.

In certain embodiments, the ATP detection reagent is a chemiluminescent system, including without limitation a luciferase/luciferin system. In certain embodiments, the luciferase is a surface-bound enzyme. The ATP produced by the polymerase reaction is consumed in the luciferin-luciferase reaction, resulting in the production of inorganic pyrophosphate and light. Thus, the amount of light produced is directly proportional to the amount of ATP released by the polymerase, which in turn is directly proportional to the number of ARNs incorporated into the nascent polynucleotide. In certain embodiments, the light generated by the luciferin-luciferase reaction is detected. Such detection methods are well-known and commonly employed in the art.

### Methods

The present invention provides methods for the detection or quantification of a nucleic acid target sequence, including distinguishing between genetic variations and single nucleotide polymorphisms, comprising the steps of: contacting a sample suspected of containing the target sequence in a reaction mixture as described above; and measuring the change in signal from the ATP detection reagent(s), where the level of change is proportional to the amount of target sequence present in the sample. Targets that can be specifically detected and/or quantified with this method include, but are not limited to, genomic DNA, plasmid DNA, cloning inserts in plasmid DNA, mRNA transcripts, ribosomal RNA, miRNA, noncoding RNA, viral RNA or DNA, PCR amplicons, restriction fragments, synthetic oligonucleotides, as well as any other nucleic acids and oligonucleotides.

In such assays, a change in signal that results from the presence of released ATP, e.g. a fluorescent signal, light, etc. is generated by the DNA polymerization from the presence of the target polynucleotide in the sample. The signal is monitored and quantified with detectors, such as fluorescence spectrophotometers, microplate readers, UV lamps, PCR, commercial systems that allow the monitoring of fluorescence in real time reactions, or, in some instances, by the human eye. Where the detectable signal is light, e.g. from a luciferase based system, a wide range of lumimometer devices are commercially available for tubes, plates, multimodal plates, etc.

Assays based on detection of sequences present in individual cells may utilize fixed cells. Cells in a sample may be fixed, e.g. with 3% paraformaldehyde, and are usually permeabilized, e.g. with ice cold methanol; HEPES-buffered PBS containing 0.1% saponin, 3% BSA; covering for 2 min in acetone at −20° C.; and the like as known in the art.

Such assays may be conducted with mRNA samples obtained from a biological system under different environmental conditions, such as exposures to varying concentration of a drug candidate or mixtures of drug candidates, which can provide data on the efficacy, the safety profile, the mechanism of action and other properties of the drug candidates that are required in drug development. Alternatively, tissue samples may be probed for the presence of clinical conditions, e.g. the presence of pathogens; expression of tumor associated sequences; and the like.

In another embodiment of the invention, the probes are used to detect or quantify nucleic acid targets from genomic DNA, in order to analyze for the presence or absence of polymorphisms in the genomic DNA. The polymorphisms can be deletions, insertions, or base substitutions or other polymorphisms of the genomic DNA or mRNA. Typically the polymorphisms are single nucleotide polymorphisms (SNPs), gene rearrangements, allelic variants; and the like.

### Allele Specific Detection

In some embodiments of the invention, the presence of a specific allele in a sample is detected. In some such methods, two or more reaction mixtures as described above are used, where each reaction mixture comprises a primer specific for one allele. The allelic variation may include, without limitation, single nucleotide polymorphisms, gene rearrangements, single nucleotide deletions, single nucleotide insertions, etc. Alternatively, a single reaction containing one allele specific primer is used, and compared to a reference sample. Primers are designed to be complementary to one or more of the allelic forms, where the terminal 3′ nucleotide of the primer is designed to be specific to a position of variation.

A sample comprising polynucleotides suspected of containing an allelic variant is divided into each of the two or more reaction mixtures, which reaction mixtures differ in the sequence of the primer. After contacting the polynucleotide sample with the reaction mixtures; the change in signal from the ATP detection reagent(s) is measured, where the level of change is proportional to the amount of allele specific target sequence present in the sample. The change in ATP signal may be compared between the reaction mixtures, or compared to a reference value, e.g. a control value (e.g., a mean and standard deviation) from a polynucleotide sample of known allelic sequence. One skilled in the art will recognize that there are many statistical methods that may be used to determine whether there is a significant difference in values.

The level of ATP present in a sample that contains at least one allele specific target is increased relative to the level of ATP released where the allele specific target is absent. The increase may be at least about 50%, at least about 1-fold, at least about 2-fold, at least about 3-fold, or more. In some embodiments a qualitative analysis is made, e.g. as to whether an allele is absent or present. In some embodiments a quantitative analysis is made, where the analysis provides information regarding the level of an allele that is present in a polynucleotide sample. Such analysis may be used, for example, in a heterogeneous sample, such as a tumor sample, a mixed population, etc. Such analysis may also be used to determine if an individual is heterozygous for the alleles of interest.

### Kits

Also provided are kits for practicing the subject methods. The kits according to the present invention may comprise at least a combination of ARN and dNTP reagents in concentrations and ratios suitable for use in the methods described herein. For example and without limitation, a composition can be provided containing a stock solution of all four deoxynucleotides, where each deoxynucleotide is provided either as a native dNTP, or as an ARN and where at least one ARN is present. In some such embodiments the ARN is one or both of dAppppA and dGppppA. In some embodiments, the four deoxynucleotides are provided as three ARNs, and one native dNTP. In some embodiments, all four ARNs are present. For any given base, the stock solution will contain a native dNTP or an ARN, but not both carrying the same nucleobase.

A kit may further include a polymerase or reverse transcriptase. In some embodiments the polymerase is E. coli Pol I or a derivative or fragment thereof, e.g. Klenow fragment, Kf exo⁻ A kit may further include reagents for detecting ATP, including, but not limited to one or both of: (a) an ATP-responsive fluorescent dye; (b) a luciferase and luciferin. A kit may further include additional reagents employed in the methods of the invention, e.g., buffers, nuclease inhibitors, etc. In certain embodiments, the kits will further include instructions for practicing the subject methods or means for obtaining the same (e.g., a website URL directing the user to a webpage which provides the instructions), where these instructions may be printed on a substrate, where substrate may be one or more of: a package insert, the packaging, reagent containers and the like. In the subject kits, the one or more components are present in the same or different containers, as may be convenient or desirable.

A kit may include a primer to initiate polymerase synthesis on a specific DNA or RNA target. This primer may be specific to single nucleotide variations (an allele specific primer), in which the 3′ terminal nucleotide is complementary to one genetic variant but mismatched to another single nucleotide variant. Two primers (one for each single nucleotide variant) may be included. A kit may also include a circular template for amplification; it may also include a primer for branched RCA.

The various reagent components of the kits may be present in separate containers, or may all be precombined into a reagent mixture for combination with samples. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXPERIMENTAL

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### Example 1

### ATP-Releasing Nucleotides: Linking DNA Synthesis to Luciferase Signaling

A new strategy is provided to produce luminescence signals from DNA synthesis by designing chimeric nucleoside tetraphosphate dimers in which ATP, rather than pyrophosphate, is the leaving group. We describe the synthesis of ATP-linked nucleotides (ARNs) as derivatives of the four canonical nucleotides. We find that the four are good substrates for DNA polymerase, with Kₘ values averaging 13-fold higher than those of natural dNTPs, and k_{cat} values within 1.5-fold of those of native nucleotides. Importantly, ARNs are found to yield very little background signal with luciferase. DNA synthesis experiments show that the ATP byproduct can be harnessed to elicit a chemiluminescence signal in the presence of luciferase. Using a polymerase and a primer complementary to a genetic target together with the chimeric nucleotides, target DNAs/RNAs trigger the release of stoichiometrically large quantities of ATP, allowing sensitive isothermal luminescence detection of nucleic acids (genetic targets) as diverse as phage DNAs and short miRNAs.

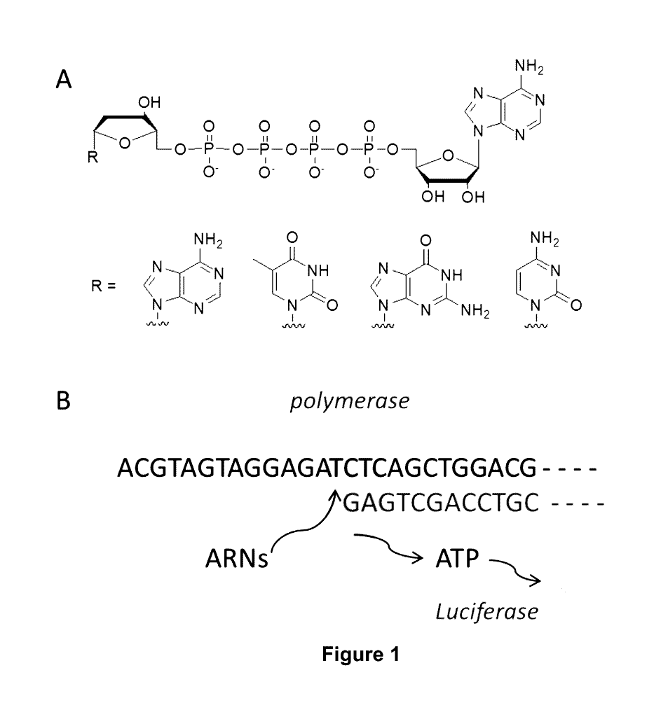

Here we describe the design and application of ATP-linked nucleotides (ARNs, FIG. 1) as reporters of DNA synthesis. These tetraphosphate-bridged chimeric RNA-DNA dinucleotides are employed sequentially as substrates for DNA polymerases and for luciferase. In this design, DNA polymerase uses the ARNs to copy a target strand, releasing one equivalent of ATP for every deoxynucleotide incorporated. In a subsequent reaction, luciferase can then process the ATP products to generate light signals in the presence of luciferin. In principle, the longer the target nucleic acid molecule, the more signals are generated, thus giving the possibility of high sensitivity. Although dimeric polyphosphate-linked nucleotides are known in the literature, the ATP-linked chimeric nucleotides have not been studied previously.

Tetraphosphate-bridged DNA-DNA dinucleotides have been the subject of a report testing them as substrates for DNA polymerases. Tetraphosphate-linked riboribodinucleotides have been studied more widely, as inhibitors of kinases, endoribonuclease, IMP dehydrogenase, adenylosuccinate synthetase, and poly(ADP-ribose) polymerase. In addition, the dinucleoside tetraphosphate Up4U has been approved as a drug for the treatment of dry eye syndrome. Despite these precedents, we know of no literature reports of chimeric ribo-deoxy tetraphosphate dinucleotides.

Thus we undertook the current study; a priori it was not known (1) whether an efficient synthesis of the chimeric nucleotides could be developed; (2) whether DNA polymerases would readily accept the dinucleotides without interference from the chemically similar ATP group at the opposite end; (3) whether luciferase might accept the dinucleotides as substrates, thus bypassing the polymerase and short-circuiting this concept; (4) what enzymes and conditions would yield optimal signals; and (5) what sensitivity the approach might have in reporting on nucleic acid targets.

We report a one-pot synthetic procedure that produces each of the four chimeric ARNs from ATP and deoxynucleoside monophosphates. We find that the chimeric dinucleotides are in fact efficient substrates for DNA polymerase, but are inefficient with luciferase, thus minimizing background signal. These properties enable their use in luminescence reporting of DNA polymerase activity, including sensitive detection of DNA and RNA target analytes.

For reporting on DNA polymerase activity with all possible sequences, a full set of four chimeric ATP-linked dinucleotides is needed. We prepared these by modifying a published procedure used previously for end-labeled nucleoside tetraphosphates (Sims et al. Nat. Methods 2011, 8, 575-580.) Tetra- or tri-alkylammonium salts of standard deoxynucleoside monophosphates (dNMPs) were activated with carbonyldiimidazole and then reacted with the alkylammonium salt of 5′-ATP to produce the desired chimeric dimers. These ARNs were purified by HPLC and ion exchange chromatography, yielding products as lyophilized powders in 42-60% yields.

Our initial studies were directed at testing whether these modified nucleotides would be active as substrates for a DNA polymerase. We carried out experiments of primer extension on short synthetic primer/template duplexes (FIG. 2; 1 μM) in the presence of a standard polymerase (Klenow fragment of DNA polymerase I 3′-exonuclease deficient variant, Kf exo⁻). We supplied one ARN at a time (20 μM) to its complementary template for one hour; if synthesis were successful it should generate up to ˜20 μM ATP as by-product of the reaction. We removed a small aliquot of the polymerase reaction and measured luminescence from this aliquot containing ATP in a commercial luciferase+luciferin reaction buffer over one hour (FIG. 2B). The results showed that signals were clearly generated for each of the four DNA templates, resulting in about equal intensities except for the G20 template sequence, which generated a moderately smaller signal. In this latter case we hypothesize the presence of intra- or inter-molecular G quadruplex structures that may inhibit the polymerase, possibly explaining the diminishment of ATP signal. In all four cases, signals were considerably (13-33-fold) higher than background in which primer/template DNA was omitted.

Next we tested sequence selectivity of the chimeric nucleotides, evaluating sixteen combinations of ARNs with the four DNA sequences (FIG. 2C and FIG. 5). In all cases, the correct nucleotide/target sequence combinations yielded much higher signals than incorrect combinations, showing clear nucleotide/template base selectivity. Interestingly, the adenosine ribonucleotide moiety of these chimeras was not noticeably misincorporated by the Kf polymerase, as evidenced by the lack of enhanced signal on the T₂₀ template sequence for dTppppA, dCppppA, and dGppppA. The main background signal appeared from experiments containing dCppppA; subsequent experiments (below) revealed that this arises primarily from a small degree of background reaction of the chimeric nucleotide preparation with luciferase rather than from misincorporation by DNA polymerase. Thus we conclude that the adenosine linkage at the terminus of these deoxynucleotides does not greatly diminish either their efficiency or selectivity with a common DNA polymerase. For the ARNs to be useful in detecting naturally occurring DNA or RNA targets, they need to function with high efficiency and low background. Moreover, the utility of ARNs would be increased if they could be accepted as substrates by variety of DNA polymerase enzymes. With these issues in mind, we carried out a number of experiments to further characterize these chimeric nucleotides as enzyme substrates.

First, it is important to determine whether ARNs can directly act as efficient luciferase substrates. If this were the case, one would observe strong signals whether or not a DNA polymerase or a template DNA were present, nullifying their utility in reporting on DNA synthesis. Thus we compared luciferase signals in the absence of DNA or polymerase, supplying each of the ARNs in separate experiments, and comparing the luciferase/luciferin luminescence signal to that with native ATP. The results showed (FIG. 6) that the ARNS are poor substrates for luciferase, yielding from 50 to >300-fold lower signals at the standard luciferase 5 min time point than ATP. Overall, we conclude that background signals from the ARNs are quite low, and that judicious choice of ARN can be used to suppress remaining background (see below).

Next we performed experiments to quantify the efficiency of ARNs as DNA polymerase substrates. We used published analytical high-resolution gel-based methods to evaluate steady state kinetics of the four nucleotides as substrates during single nucleotide incorporation experiments, with Kf exo⁻. We performed similar measurements with the natural dNTPs for comparison. The data are summarized in Table 1 (see also FIG. 8); the experiments reveal that the ARNs are substrates with efficiencies moderately less than those of native dNTPs. Kₘ values average 2.5 μM, higher than those of natural nucleotides, which have values averaging 0.2 μM. Values for k_{cat}, on the other hand, are very similar for the chimeric nucleotides (7.7 min⁻¹) and native dNTPs (11.7 min⁻¹). Thus, although somewhat higher concentrations may be required to achieve near maximum velocities for ARNs, the maximum rates for polymerase incorporation are expected to be nearly the same as those of native nucleotides. The most efficient ARN (compared to its native congener) is dAppppA, which exhibits k_{cat}/Kₘ value only 5-fold less than that of dATP, while the least efficient is dGppppA, which is less efficient than dGTP by a larger factor of 70 (with most of this factor in the Kₘ term).

TABLE 1

| | k_{cat} | Kₘ | k_{cat}/Kₘ |
|---|---|---|---|
| dNTP | (min⁻¹) | (μM) | (μM⁻¹min⁻¹) |
| dGTP | 15.4 ± 0.5 | 0.11 ± 0.01 | 140 |
| dGppppA | 7.1 ± 0.5 | 3.5 ± 0.4 | 2.0 |
| dCTP | 14.0 ± 0.4 | 0.07 ± 0.01 | 200 |
| dCppppA | 12.9 ± 0.1 | 3.0 ± 0.2 | 4.3 |
| dATP | 8.6 ± 0.3 | 0.35 ± 0.05 | 25 |
| dAppppA | 7.1 ± 0.2 | 1.3 ± 0.4 | 5.5 |
| dTTP | 8.7 ± 0.3 | 0.24 ± 0.06 | 36 |
| dTppppA | 3.7 ± 0.1 | 2.2 ± 0.6 | 1.7 |

Steady-State DNA Polymerase Efficiency with Chimeric ATP-Releasing Nucleotides, with Kf Exo⁻.

Next we explored the question of whether other DNA polymerases can accept ARNs as substrates. We tested a range of DNA polymerases and reverse transcriptases, and carried out primer extension studies (FIG. 7A) and luciferase measurements of activity (FIG. 7B) on a short linear template. The data shows that several polymerases successfully extend primers exclusively using these chimeric nucleotides. Measurement of signal after primer extension reactions showed that a number of DNA polymerases and reverse transcriptases do yield luciferase signals over background. Interestingly, the strongest signals were seen with Kf pol with exonuclease activity, suggesting that proofreading activity may enhance signals by requiring re-synthesis of existing base pairs. However, very strong exonuclease activity (T4, T7 pols) rapidly degrades the primer, thus diminishing signal by preventing initiation. Overall, we conclude that multiple DNA polymerases and reverse transcriptases can process these modified substrates and generate substantial ATP signals.

The preliminary data demonstrate the use of ARNs in reporting on varied classes of DNA or RNA targets. The above experiments revealed that different ARNs yield greater or smaller amounts of background signal; moreover, different ARNs are variably active as DNA polymerase substrates. Although in principle one might use all four ARNs exclusively in DNA synthesis for detecting a target, one might conceivably enhance signal-to-background ratio by using a smaller subset of ARNs in combination with native dNTPs.

To explore this possibility, we tested varied combinations of ARNs in primer extension experiments with single-stranded phage M13 DNA, evaluating signals via our standard subsequent luciferase reaction. The data are shown in FIG. 9. The experiments revealed that all four ARNs could indeed be used simultaneously to copy a DNA target of complex sequence, generating a robust signal (FIG. 9, lane 1). However, replacement of dCppppA with dCTP yielded a ˜10% higher signal, rather than 25% lower as expected from the stoichiometry assuming an equal length of DNA synthesized. Similarly, replacing both dCppppA and dTppppA yielded yet higher signal (lane 3). Further signal increase was not realized by also removing dGppppA and replacing it with dGTP (lane 4); thus, the combination of two ARNs with two native ones yielded the strongest signal. Measuring the background for these combinations (with no DNA target) showed that omission of two of the nucleotides also lowered background signal by several-fold (compare lane 3c with 1c).

The above experiments establish that chimeric ATP-linked nucleotides can be used to generate luminescence signals via luciferase when a DNA polymerase has been active on a nucleic acid template. This signaling can be used to detect the presence of a given genetic target. Interestingly, given enough time, a longer template is expected to yield more signals than a short one, since there are stoichiometrically greater quantities of nucleotides consumed (and ATP generated) per molecule of target. This concept leads to multiple predictions about nucleic acid target detection: first, that long biological nucleic acids can be detected quite sensitively on a per molecule basis; second, that circular templates could generate relatively large signals due to the polymerase progressing more than once around the template; and third, that short genetic targets would yield only small signals when used as templates, but could generate larger signals if employed instead as primers on long or circular templates. We explored these issues in subsequent experiments with two classes of genetic targets: bacteriophage DNA and miRNA.

Bacteriophage M13mp18 DNA is a single-stranded, circular DNA 7249 nt in length. We envisioned the use of a phage specific primer with Kf polymerase and two ARNs (see above) for detection of this nucleic acid target. Initial tests with three different primers (FIG. 3A) showed that two DNA primers complementary at distinct sites in the phage each yielded identical amounts of signal with 1 nM phage DNA, while a noncomplementary primer (sense rather than antisense in complementarity) yielded little signal, the same as the control lacking DNA. Similarly, a primer fully complementary except mismatched at the three 3′-terminal nucleotides also yielded approximately background levels of signal, consistent with the need for 3′ end priming to initiate reaction (FIG. 3A). Experiments with 10 vs 24 h polymerase reactions (Kf pol) showed significant enhancement between these times, confirming that the Kf DNA polymerase remained active for a long period, as expected on this circular target. Experiments at shorter times (50 fmol target) confirmed that there was significant signal over background at times shorter than one hour (FIG. 11). Next we evaluated the limit of detection, carrying out full 24 h polymerase reactions with 20 pM dAppppA, dGppppA, dTTP and dCTP, and diluting the DNA over several log units range of concentration (FIG. 3B). The data show that 5 attomoles (5×10⁻¹⁸ moles) of phage M13 DNA could be reproducibly detected over background.

Next we turned our attention to detection of miRNAs, an important class of short nucleic acid targets. Several families of miRNAs have been linked to cancer, and so developing methods for detecting these small single-stranded RNAs has been an active research goal. The let-7 family of miRNAs in particular has been shown to play significant roles in ovarian, prostate, liver and pancreatic cancer. Since miRNAs are short, polymerase chain reaction (PCR) cannot be carried out on the unmodified target. Additional steps (such as ligation) are needed to modify miRNAs for PCR-based detection, and so simpler approaches merit investigation. In the present approach, detecting these RNAs as polymerase templates with ARNs would not be expected to yield high sensitivity because of their short length. For that reason, we instead took the approach of employing them as primers, using a small circular DNA template complementary to the target let-7a miRNA. In this strategy, RCA is carried out, primed by the miRNA on the circular DNA. (see Jonstrup et al. RNA 2006, 12, 1747-1752; Zhou et al. Nucleic Acids Res. 2010, 38, e156; Deng et al. Angew. Chem. Int. Ed. 2014, 53, 2389-2393; Liu et al. Anal. Chem. 2013, 85, 7941-7947; Cheng et al. Angew. Chem. Int. Ed. 2009, 48, 3268-3272; Harcourt and Kool, Nucleic Acids Res. 2012, 40, e65).

Although the circle is short (50 nt), polymerases are known to proceed hundreds or thousands of times around such templates, thus consuming many thousands of nucleotides per miRNA molecule. This can be extended yet further with hyperbranched RCA, by supplying a DNA primer complementary to the initial RCA multimeric product. Isothermal detection of miRNAs via rolling circle templates has been reported previously, using templated fluorogenic chemistry or DNA-binding fluorescent dyes to report on the long DNA products.

Experiments in the presence of ARNs showed that the 22mer let-7a RNA could indeed prime DNA synthesis by the highly processive 4)29 DNA polymerase, using the 50 nt circular DNA complementary to the miRNA as template. Signal was observed above background for reactions as short as 1 h (FIG. 12), but longer polymerase reactions produced considerably greater signals. To measure sensitivity, reactions were carried out at 30° C. with 10 nM circular DNA template and varied concentrations of miRNA, with 50 μM dAppppA, dGppppA, dTTP and dCTP, and allowing the polymerase step to proceed for 24 h for maximum signal. Subsequent luminescence detection showed signals above background for as little as 10 attomoles of target RNA (FIG. 4A). For comparison to the ARN/luciferase detection reaction, we tested the use of DNA-binding fluorescent dyes for detecting product in otherwise identical branched RCA reactions with let-7a RNA. The results are shown in FIGS. 4B,C; limits of detection for the two dyes were 0.05-0.5 fmol of the miRNA, showing ˜1-2 orders of magnitude less sensitivity than ARN detection with luciferase. Controls with varied sequence (DNA or RNA targets) confirmed selective signaling for the let-7a target; for example, a 3′-terminally mismatched target showed diminished signal (FIG. 4D, let-7aM), as did a naturally occurring variant with a mismatch 4 nt from the 3′ end (let-7i). Targets mismatched near the center, however, showed lower selectivity, as expected since the target 3′ end remains complementary to the circular DNA (FIG. 13). Nevertheless, a single nucleotide mismatch (let-7e sequence) did produce a measurable diminishment of signal.

Taken together, our experiments have shown that ATP linked deoxynucleotides act as good polymerase substrates and yield little background reaction with the luciferase enzyme. These facts enable these chimeric nucleotides to be employed in sensitive detection of nucleic acid target molecules. The method is isothermal and simple, requiring only one DNA probe and a standard DNA polymerase. No labelling of probe or target is required. The strategy is versatile, detecting DNA or RNA, and short or long targets can be sensitively detected with judicious design of primer or circular template. The separation of the luciferase signaling reaction from the polymerase reaction₃₂ allows one to measure signals via 96-well plate reader at a convenient time after multiple polymerase reactions have been carried out.

The sensitivity of the ARN/luciferase method compares well to literature methods for isothermal detection of nucleic acids. For example, isothermal miRNA detection via rolling circle amplification reactions has been reported to achieve sensitivity of 30 attomoles (SYBR Green I dye) or 2 fmol (with templated chemistry). Our own experiments confirm an advantage of 1-2 orders of magnitude over DNA-binding fluorescent dyes. Although possibly not more sensitive than PCR-based approaches to miRNA detection, the current method is simpler, requiring fewer primers and enzymes, fewer steps, and no thermal cycling equipment. Limitations of the current approach arise from its inherent design; for example, it is difficult in the current strategy to detect single-nucleotide variants in a miRNA target if the polymorphism occurs near the center or 5′ end, since only a complementary 3′ terminus is needed to prime synthesis. Further design modifications will be needed to address this and related issues.

ARNs may also find use in reporting on multiple classes of biomolecules. Since ATP acts as an energy source in multiple biological processes, ARNs also finds use in polymerase-mediated activation of enzymatic activities other than luciferase.

### Materials and Methods

Synthesis of ATP-linked deoxynucleoside tetraphosphates. The chimeric nucleotide dimers were prepared from alkylammonium salts of ATP and the corresponding deoxynucleoside 5′-monophosphates, in a modification of a published procedure. The phosphate-phosphate bond was formed after activation with carbonyldiimidazole in DMF solvent.

Primer extension and luciferase signaling with short linear test templates. 36mer DNA templates were separately annealed with a 16mer primer (FIG. 3A) in a polymerase buffer. A standard 20 μL polymerase reaction contained: 1 μM primer-template duplex, 20 μM chimeric ATP-linked nucleotides, and 1 μL polymerase stock (from manufacturer). After 1 h reaction at 37° C., 5 μL of this solution was added to 95 μL luciferase reaction solution (prepared as per the ATP determination kit) in a 96 well plate. The bioluminescence signal was recorded by microplate fluorometer (Fluoroskan Ascent, Thermal).

Detection of circular phage M13 DNA. 100 nM M13mp18 single-stranded DNA was annealed with primer A1 SEQ ID NO:4 (5′-GCAGGTCGACTCTAGAGGAT-3′), using procedures described above. The annealed M13mp18 single stranded DNA/primer complex was diluted to the desired concentration (10 nM to 1 μM). Standard 20 μL polymerase reactions contained: 2 μL appropriate concentration of annealed M13mp18 single-stranded DNA/primer complex, 20 μM each of chimeric ATP-linked nucleotides (dAppppA and dGppppA) and 20 μM natural nucleotides (dTTP and dCTP), 1× polymerase reaction buffer and 1 μL Klenow fragment polymerase. Reactions were incubated at 37° C. for 10 h or 24 h, then denatured at 65° C. for 15 min. The detection of ATP products was carried out by adding 5 μL polymerase reaction solution to 95 μL luciferase reaction solution (prepared as instructed by the manufacturer) in a 96 well plate. Luminescence was monitored by microplate fluorometer over 2 h. See Supporting Data file for detailed methods and additional experimental data.

Detection of miRNA. Standard 20 μL polymerase reactions contained: 2 μL appropriate concentration of miRNA (5 μM to 10 nM), 10 nM small circular ODN, 50 μM each of chimeric ATP-linked nucleotides (dAppppA and dGppppA) and 50 μM natural nucleotides (dTTP and dCTP), 1 μL 10 μM primer stock (SEQ ID NO:3, 5′-TCTCTCGTGCAGACT-3′), 1× polymerase reaction buffer and 1 μL φ29 DNA polymerase. Reactions were incubated at 30° C. for 24 h, then denatured at 65° C. for 15 min. The bioluminescence signal was measured as described in detection of M13 DNA.

Reagents and starting materials for chemical syntheses were obtained from commercial suppliers (Sigma-Aldrich or Alfa Aesar) unless otherwise indicated. DNA and RNA oligonucleotides were obtained from Integrated DNA Technologies (IDT, Coralville, Iowa, USA) used as provided. Purity was judged to be >95% by analytical gel analysis. M13mp18 single-stranded DNA was purchased from New England BioLabs (Ipswich, Mass.). The 50-nt let-7 circular oligonucleotide (sequence SEQ ID NO:5, 5′-TACTACCTCATCATTTCTCTCGTGCAGACTCGGACTTTAAC TATACAACC-3′) was prepared as described previously (Harcourt and Kool, Nucleic Acids Res. 2012, 40, e65). [γ-³²P]ATP was obtained from Perkin Elmer (Piscataway, N.J.). Enzymes were purchased from New England BioLabs (Ipswich, Mass.) unless otherwise specified. ATP Determination Kit (A22066) from Life Technologies (Invitrogen) was used for the bioluminescence assay. SYBR Gold (10,000× Concentrate in DMSO) was purchased from ThermoFisher. EvaGreen Dye, 20× in water was purchased from Biotium.

Preparation of the tetrabutylammonium or tributylammonium salts of nucleoside monophosphates. The sodium salts of ATP, dAMP, dTMP, and dCMP were dissolved in distilled deionized water and converted into their free acids using a Dowex-50W ion exchange column (H+ form), titrated to pH 7.0 with a diluted solution of tetrabutylammonium hydroxide, and then lyophilized twice to white powders. dGMP was converted via a different method due to apparent decomposition after titration with tetrabutylammonium hydroxide. The aqueous solution of dGMP disodium salt dihydrate was applied to a Dowex-50W column (pyridinium form). The eluate was collected in a flask containing tributylamine in ethanol solution. The resulting solution was concentrated and then lyophilized twice, yielding a white powder. The lyophilized powder of all the nucleotides were coevaporated with anhydrous DMF twice and kept under high vacuum for 3 h before the subsequent coupling reaction (below).

General method for the synthesis of ATP-linked nucleotides. The tetrabutylammonium salt of ATP (60 mg, 40 μmol) was dissolved in 1 mL anhydrous DMF. To the solution, carbonyldiimidazole (CDI, 25.8 mg, 160 μmol) was added, and the mixture was stirred at room temperature for 5 h, after which 50 μL MeOH was added to quench the reaction. All solvents were removed under high vacuum and the residue redissolved in 1 mL anhydrous DMF. The desired deoxynucleoside monophosphate tetrabutylammonium or tributylammonium salt in 1 mL DMF and anhydrous MgCl₂ (5 mg) were added. The mixture was stirred for 72 h at room temperature. After this, the product was precipitated by the addition of acetone (20 mL). The precipitate was washed twice with 10 mL acetone. The desired product was purified by reverse phase HPLC (RPHPLC) using a preparative C18 column using a gradient of acetonitrile and 50 mM triethylammonium acetate buffer (pH 7). Fractions containing pure product were concentrated and further purified by a DEAE Sephadex G-25 anion exchange column, eluted with 500 mM NH4HCO3. The fractions containing the desired product were pooled, concentrated and repeatedly freeze-dried to yield the final product as a white powder.

Thymidine-5′-tetraphosphate-P4-5′-Adenosine (dTppppA). Following the general procedure above, dTppppA was obtained in a yield of 55% (5.9 mg) after purification by RPHPLC and DEAE column. 1H NMR (D2O, 400 MHz, NH4+ form): δ 8.33 (s, 1H), 8.03 (s, 1H), 7.41 (s, 1H), 6.03 (t, J=4.5 Hz, 1H), 5.90 (d, J=3.8 Hz, 1H), 4.56 (brs, 1H), 4.41-4.39 (m, 2H), 4.20 (brs, 1H), 4.13-3.92 (m, 5H), 2.09-2.06 (m, 2H), 1.65 (s, 3H). 31P NMR (D2O, 162 MHz, NH4+ form): δ−10.33, −10.66, −22.20, −22.30. HRMS: calculated for C20H28N7O20P4 (M-H)−810.0345, found 810.0363.

Deoxycytidine-5′-tetraphosphate-P4-5′-Adenosine (dCppppA). Following the general procedure above, dCppppA was obtained in a yield of 45% (4.0 mg) after purification by RPHPLC and DEAE column. 1H NMR (D2O, 400 MHz, NH4+ form): δ 8.37 (s, 1H), 8.10 (s, 1H), 7.83-7.80 (m, 1H), 5.97-5.91 (m, 3H), 4.57 (brs, 1H), 4.40-4.38 (m, 2H), 4.21 (brs, 1H), 4.10 (brs, 2H), 4.03-3.99 (m, 3H), 2.24-2.19 (m, 1H), 2.10-2.03 (m, 1H). 31P NMR (D2O, 162 MHz, NH4+ form): δ−10.34, −10.43, −22.12, −22.21. HRMS: calculated for C19H27N8O19P4 (M-H)−795.0348, found 795.0351.

Deoxyadenosine-5′-tetraphosphate-P4-5′-Adenosine (dAppppA). Following the general procedure above, dAppppA was obtained in a yield of 42% (3.9 mg) after purification by RPHPLC and DEAE column. 1H NMR (D2O, 400 MHz, NH4+ form): δ 8.39 (s, 1H), 8.34 (s, 1H), 8.08 (s, 2H), 6.16 (t, J=6.0 Hz, 1H), 5.80 (d, J=8.0 Hz, 1H), 4.52-4.49 (m, 1H), 4.38-4.34 (m, 1H), 4.32-4.30 (m, 1H), 4.25-4.20 (m, 2H), 4.15-4.08 (m, 4H), 2.40-2.36 (m, 2H). 31P NMR (D2O, 162 MHz, NH4+ form): δ−10.07, −10.16, −22.45, −22.54. HRMS: calculated for C20H27N10O18P4 (M-H)−819.0461, found 819.0469.

Deoxyguanosine-5′-tetraphosphate-P4-5′-Adenosine (dGppppA). Following the general procedure above, dGppppA was obtained in a yield of 60% (7.7 mg) after purification by RPHPLC and DEAE column. 1H NMR (D2O, 400 MHz, NH4+ form): δ 8.27 (s, 1H), 8.00 (s, 1H), 7.81 (s, 1H), 5.94 (t, J=8.0 Hz, 1H), 5.85 (d, J=8.0 Hz, 1H), 4.54 (brs, 1H), 4.50-4.48 (m, 1H), 4.37-4.35 (m, 1H), 4.20-4.19 (m, 1H), 4.13-4.11 (m, 2H), 4.04-4.00 (m, 3H), 2.51-2.44 (m, 1H), 2.29-2.23 (m, 1H). 31P NMR (D2O, 162 MHz, NH4+ form): δ−10.21, −10.31, −22.06, −22.16. HRMS: calculated for C20H27N10O18P4 (M-H)−835.0410, found 835.0421.

Initial primer extension studies of chimeric nucleotides with Klenow fragment (exo-) DNA polymerase. 36 mer ODNs (SEQ ID NO:6, 5′-(T)20TCATCCGCTAGCTCGA-3′; SEQ ID NO:7, 5′-(A)20TCATCCGCTAGCTCGA-3′; SEQ ID NO:8, 5′-(G)20TCATCCGCTAGCTCGA-3′; SEQ ID NO:9, 5′-(C)20TCATCCGCTAGCTCGA-3′) were separately annealed with a 16mer primer (SEQ ID NO:10, 5′-TCGAGCTAGCGGATGA-3′) by being heated to 85° C. and cooled slowly to room temperature, in NEB buffer 2. A standard 20 μL polymerase reaction contained: 1 μM anealed primer, 20 μM single chimeric ATP-linked nucleotides, 1× polymerase reaction buffer (from manufacturer) and 1 μL polymerase (from manufacturer). After 1 h incubation at 37° C., 5 μL of this reaction solution was added to 95 μL luciferase reaction solution (prepared as per the ATP determination kit) in a 96 well plate. The bioluminescence signal was recorded at 1 min intervals over 1 h by microplate fluorometer (Fluoroskan Ascent, Thermal).

Primer extension assays with varied polymerases and reverse transcriptases. The 13 mer primer (SEQ ID NO:11, 5′-CTAGGATCATAGC-3′) was end-labeled with T4 polynucleotide kinase and [γ-³²P] ATP at the 5′ end following the manufacturer's protocol. The 20 mer ODN (SEQ ID NO:12, 5′-ATGGCGTGCTATGATCCTAG-3′) was then annealed with the 5′-³²P-labeled 13mer primer as described above. Standard 20 μL polymerase reaction contained: 0.1 μM annealed primer, 20 μM chimeric ATP-linked nucleotides, 1× reaction buffer and 0.5 μL polymerase or reverse transcriptase. The reaction mixture was incubated at 37° C. for 1 h (except φ29 polymerase at 30° C. and Taq polymerase at 65° C.). The reaction was terminated with an equal volume of formamide gel loading buffer (95% formamide, 20 mM EDTA, 0.05% xylene cyanol and bromophenol blue). The products were resolved on 20% denaturing polyacrylamide gels containing 8 M urea, and gel band intensities were quantified using a Typhoon 9410 Imager (Amersham Biosciences Co.)

Steady-state kinetics measurements. Steady-state kinetics assays were performed following previously published procedures (Swanson et al. Biochemistry 2011, 50, 7666-7673). The 13mer primer (SEQ ID NO:13, 5′-CTAGGATCATAGC-3′) was end labeled with T4 polynucleotide kinase and [γ-³²P]-ATP following the manufacturer's protocol. The 20mer template ODNs (SEQ ID NO:14, 5′-ATGGCGNGCTATGATCCTAG-3′, N=A, T, C, G) were annealed with 5′-³²P-labeled 13mer primer as described above. The annealed primer-template duplex (0.05 μM) was incubated with Klenow fragment exo⁻ polymerase at 37° C. for 5 min in the presence of individual dNTPs or chimeric ATP-linked nucleotides at varied concentrations.

The parameters were adjusted to result in extents of reaction of 20% or less to maintain initial velocity conditions. The reaction was terminated with an equal volume of formamide gel loading buffer (95% formamide, 20 mM EDTA, 0.05% xylene cyanol and bromophenol blue). Extension products were separated on 20% denaturing polyacrylamide gels containing 8 M urea. Gel band intensities of the primer and its extension products were quantified using a Typhoon 9410 Variable Mode Imager. Quantitative imaging of bands was carried out using Image J software to determine the fraction of primer extension. The velocity was plotted as a function of dNTP (or ARN) concentration and fit with the Michaelis-Menten equation to obtain the kinetic parameters, Vₘₐₓ and Kₘ. Reactions were performed three times and the mean (±standard deviation) of Vₘₐₓ and Kₘ are reported. The k_{cat} values were calculated by dividing the the Vₘₐₓ with the concentration of polmerase used. The efficiency of nucleotide incorporation was calculated by the ratio K_{cat}/Kₘ.

Luciferase detection of M13mp18 single-stranded DNA using chimeric nucleotides. 100 nM M13mp18 circular single-stranded DNA was annealed with primer A1 (SEQ ID NO:4, 5′-GCAGGTCGACTCTAGAGGAT-3′) using procedures described above. The annealed M13mp18 single-stranded DNA/primer complex was diluted to appropriate concentration (10 nM to 1 μM). Standard 20 μL polymerase reaction contained: 2 μL appropriate concentration of annealed M13mp18 single-stranded DNA/primer complex, 20 μM each of chimeric ATP-linked nucleotides (dAppppA and dGppppA) and 20 μM natural nucleotides (dTTP and dCTP), 1× polymerase reaction buffer and 1 μL Klenow fragment polymerase. Reactions were incubated at 37° C. for 10 h or 24 h, then denatured at 65° C. for 15 min. The detection of ATP products was carried out by adding 5 μL polymerase reaction solution to 95 μL luciferase reaction solution (prepared as instructed by the ATP determination kit) in a 96 well plate. Luminescence was monitored by microplate fluorometer over 2 h.

To test varied primers, 1 nM primer A1 (SEQ ID NO:4, 5′-GCAGGTCGACTCTAGAGGAT-3′), A2 (SEQ ID NO:15, 5′-GGAAACAGCTATGACCATG-3′), S1 (SEQ ID NO:16, 5′-GTAAAACGACGGCCAGTG-3′) or A1M (SEQ ID NO:17, 5′-GCAGGTCGACTCTAGAGCTC-3′) were mixed with 1 nM M 13mp18 single-stranded DNA in standard 20 μL polymerase reaction mixture as above. Reactions were incubated at 37° C. for 10 h, then denatured at 65° C. for 15 min. The detection of ATP products was carried out as described above.

Detection of let-7a miRNA with branched RCA and ARNs followed by luciferase. Standard 20 μL polymerase reactions contained: 2 μL appropriate concentration of miRNA let-7a (2 μM to 10 nM), 10 nM small circular ODN, 50 μM each of chimeric ATP-linked nucleotides (dAppppA and dGppppA) and 50 μM natural nucleotides (dTTP and dCTP), 1 μM primer stock (SEQ ID NO:3, 5′-TCTCTCGTGCAGACT-3′), 1× polymerase reaction buffer and 1 μL φ29 DNA polymerase. Reactions were incubated at 30° C. for 24 h or as indicated, then denatured at 65° C. for 15 min. The bioluminescence signal was measured as described in detection of M13 DNA.

Detection of miRNA after branched RCA using nucleic acid binding dyes. For branched RCA, we used 50 μM of the four natural nucleotides (dATP, dGTP, dTTP and dCTP) instead of chimeric ATP-linked nucleotides. Other conditions and reagents are the same as the experiments including chimeric ATP-linked nucleotides. For the detection of branched RCA products using SYBR Gold dye, 5 μL polymerase reaction solution was added to 95 μL SYBR Gold 1×H2O solution in a 96 well plate. After incubating for 5 min, the microplate reader was used to measure the fluorescence at 538 nm with excitation wavelength at 485 nm. For the detection using Eva Green dyes, 10 μL polymerase reaction solution was added to 190 μL 1×H2O EvaGreen dye solution. After incubation for 5 min, the fluorescence signal at 525 nm was measured by fluorometer with excitation wavelength at 500 nm.

Test of selectivity among closely related let-7 family members. Polymerase reactions were carried out with 1 nM varied let-7 family members at 37° C. for 10 h or 20 h. Reactions conditions were as in “detection of let-7a miRNA with branched RCA”, above. The let-7 family members used in this experiments are: let-7a (SEQ ID NO:18, 5′-TGAGGTAGTAGGTTGTATAGTT-3′); let-7e (SEQ ID NO:19, 5′-TGAGGTAGGAGGTTGTATAGT-3′); let-7g (SEQ ID NO:20, 5′-TGAGGTAGTAGTTTGTACAGT-3′); let-7i SEQ ID NO:21, (DNA 5′-TGAGGTAGTAGTTTGTGCTGT-3′; SEQ ID NO:22, RNA 5′-UGAGGUAGUAGUUUGUGCUGU-3′) and let-7aM (SEQ ID NO:23, 5′-UGAGGUAGUAGGUUGUAUAUGG-3′).

Test of single-tube polymerase and luciferase reactions. 10 nM annealed M13mp18 single-stranded DNA/primer complex, 20 μM each of chimeric ATP linked nucleotides (dAppppA and dGppppA), 20 μM natural nucleotides (dTTP and dCTP), and 2 μL Klenow fragment polymerase were mixed with 100 μL luciferase reaction buffer. The luminesence signals were recorded for 10 h at 1 min intervals. Experiments revealed a substantial background signal from Kf polymerase with ARNs in the presence of luciferase with no target DNA present (red trace, FIG. 14).

### Example 2

### Amplified Luciferase Detection of Single Nucleotide Mutations or Polymorphisms in Messenger RNAs

Mutations and polymorphisms in the BRAF gene are highly correlated to response to treatment of melanoma. In particular, the BRAF V600E mutation is routinely measured in melanoma patients (and in patients with other cancers as well) as part of diagnosis and treatment decisions. Current methods for measuring this mutation commonly use polymerase chain reaction (PCR). Here we describe the use of ATP-releasing nucleotides (ARN) along with allele-specific primer (ASP) designs to detect single nucleotide variations in RNAs corresponding to the BRAF mRNA sequence in normal and mutant forms.

### Methods:

Allele-specific primers were 18 nt in length, and were purchased from IDT.

BRAF ASP-A (complementary to BRAF WT):SEQ ID NO:24, 5′-CACTCCATCGAGATTTCA 3′
BRAF ASP-T (complementary to BRAF M): SEQ ID NO:25, 5′-CACTCCATCGAGATTTCT-3′

E. coli on agar stabs were ordered from Addgene containing BRAF wild type (VVT) and BRAF V600E mutant (MUT) versions of the gene. The plasmids were isolated from the E. coli and polymerase chain reaction (PCR) was performed separately on each plasmid to generate double-stranded DNA amplicons containing 500 nucleotide pairs of the BRAF gene, and an additional 20 base pairs for the T7 RNA promoter sequence. RNA transcription was performed with T7 RNA polymerase to generate single-stranded RNA copies of the WT and MUT BRAF sequences, which were subsequently isolated.

The SNP detection reactions were performed in a 20 μL microplate well containing 100 nM RNA target, 1 μM allele-specific probe, 20 μM ATP-releasing nucleotides, and Maxima H Minus reverse transcriptase for 30 minutes at 37° C.

Luminescence was detected with a Varioskan lumimometer using a commercial ATP glow assay (Promega) containing the luciferase enzyme in a 384-well plate.

### Experimental Design:

Allele-specific primers were designed to be complementary to wild-type (VVT) and mutant (MUT) BRAF mRNA, such that the last (3′) nucleotide is positioned exactly opposite the position of variation. Primers were 18nt in length; sequences are given in Methods. The experimental design is based on the known activity of polymerase enzymes, which are much more efficient at extending primer termini that are correctly matched than termini that are mismatched. In the BRAF V600E mutation, the nucleotide at position 1799 is normally a U, but in the mutant, it is A. Thus a primer terminating with 3′A will be complementary to the VVT RNA, but will yield an unfavorable A-A mismatch with the MUT RNA.

The second aspect of this methodology is the amplification. Since our targets are relatively long RNAs, we hybridize primers at the allelic site being queried. Supplying all four ARNs will then enable a reverse transcriptase enzyme to proceed from the primer end, making DNA all along the RNA target until its end is reached. Every ARN nucleotide addition releases a molar equivalent of ATP, which can subsequently be detected very sensitively. Since the target RNA may extend far downstream of the primer end, dozens, or even hundreds or thousands, of equivalents of ATP will be produced per molecular strand of RNA target. With correctly matched primer, thousands of equivalents of ATP will be produced, whereas with a mismatched primer, much lower signal will result, as the enzyme proceeds poorly past this mismatch. Thus use of two separate primers in two experiments allows for the comparison: experiments in which one primer yields much higher signal than another allows base calling, and the SNP is correctly identified. If they yield almost the same signals, this would indicate a mixture of the two alleles in the sample, such as might occur with heterozygous patients, or with mixed cancerous and normal tissue. A negative control (with no input RNA) shows the background signal. Signals reproducibly above background indicate positive signal showing the presence of the allele being probed.

### Results:

We tested purified BRAF VVT and MUT RNAs having a length of 500 nt. We then tested the ability of the two allele-specific primers to distinguish them by luciferase signal. Luminescence signals are shown in FIG. 15. For both RNA alleles, correctly matched primer/RNA combinations produced reproducibly 3-fold more signal than mismatched ones. Thus we conclude that the ARN/ASP methods allow the easy discrimination of single nucleotide mutations. The sensitivity is high; in these experiments only 2 picomoles of RNA was present. Considerably lower amounts of RNA can also be detected and identified by this method.

Methods to detect additional SNPs are as follows. Use of ARNs and allele-specific primers for detection of single nucleotide variations (specifically those causing the V617F mutation) in JAK2 kinase RNA position 1849: Widtype=G, mutant=T. Experiments are carried out as described above, but using RNA isolated from blood or tissue from a patient.

JAK2 ASP-C (complementary to JAK2 WT): SEQ ID NO:26, 5′-ATTCTCGTCTCCACAGAC-3′
JAK2 ASP-A (complementary to JAK2 M): SEQ ID NO:27, 5′-ATTCTCGTC TCC ACA GAA-3′ Results from testing the two primers are used to identify the presence of the V617F mutation.

Use of ARNs and allele-specific primers for detection of single nucleotide variations in the ABL1 gene responsible for drug resistance against Gleevec in chronic myeloid leukemia. Mutation being queried is T3151, with RNA position 944 C (wildtype) and T (mutant). Experiments are carried out as described above, but using RNA isolated from blood or tissue from a patient.

ABL1 ASP-G (complementary ABL1 WT): SEQ ID NO:28, 5′-CGTAGGTCATGAACTCAG-3′
ABL1 ASP-A (complementary ABL1 M): SEQ ID NO:29, 5′-CGT AGG TCA TGA ACT CAA-3′ Results from testing the two primers are used to identify the presence of the T3151 mutation.

What is claimed is:

1. A method for detecting the presence of a target polynucleotide sequence in a sample comprising nucleic acids, the method comprising:
contacting the nucleic acid with a reaction mixture comprising:
at least one ATP-releasing nucleotide (ARN) having a structure 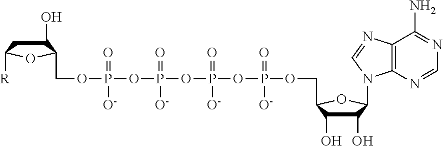
wherein R is where R is any purine or pyrimidine, or an analog thereof that retains an ability to base pair with a complementary nucleotide; and optionally dNTPs, wherein the combination of dNTPs and ARN is sufficient to provide a substrate for all bases present in the sequence of interest;
a primer or template complementary to a sequence of interest in the target polynucleotide; and
a DNA polymerase or reverse transcriptase that incorporates ARNs; and
detecting the presence of ATP released during extension of the primer or target by the DNA polymerase or reverse transcriptase.

2. The method of claim 1, wherein R is selected from adenine, thymine, guanine, and cytosine.

3. The method of claim 1, wherein the reaction mix comprises a single ARN and one or more dNTPs.

4. The method of claim 1, wherein the reaction mixture comprises two or more different ARNs.

5. The method of claim 1, wherein the reaction mixture comprises three of more different ARNs.

6. The method of claim 1, wherein the reaction mixture comprises four different ARNs.

7. The method of claim 1, wherein the reaction mix comprises one or both of dAppppA and dGppppA.

8. The method of claim 1, wherein the reaction mix comprises one or both of dAppppA and dTppppA.

9. The method of claim 1, wherein the reaction mixture comprises a DNA polymerase.

10. The method of claim 9, wherein the reaction mixture comprises a reverse transcriptase.

11. The method of claim 9, wherein the k_{cat} values for ARNs with the DNA polymerase or reverse transcriptase are within about 20-fold of those of native dNTPs.

12. The method of claim 1, wherein the reaction mixture comprises a primer complementary to the sequence of interest, of from about 8 to about 35 nt. in length.

13. The method of claim 12, wherein the complementary region of the primer is at least 90% identical to the sequence of interest.

14. The method of claim 1, wherein the primer is complementary to an allelic form, where the terminal 3′ nucleotide of the primer is specific to a position of variation.

15. The method of claim 12, wherein the primer comprises a region of non-complementarity to the sequence of interest.

16. The method of claim 1, wherein the reaction mixture comprises a template comprising a region complementary to the sequence of interest.

17. The method of claim 16 wherein the complementary region of the primer is at least 90% identical to the sequence of interest.

18. The method of claim 16, wherein the template is circular.

19. The method of claim 16, wherein the target polynucleotide is less than about 25 nt. in length.

20. The method of claim 1, wherein detecting ATP comprises the step of contacting the reaction mixture with luciferin and an ATP-dependent luciferase enzyme to produce light.

21. The method of claim 20, wherein the luciferase is added to the reaction mix after a period of time sufficient to accumulate products of the polymerization reaction.

22. The method of claim 19, wherein the luciferase is included in the initial reaction mixture.

23. The method of claim 1, wherein detecting ATP comprises the step of contacting the reaction mixture with an ATP-responsive fluorescent dye.

24. The method of claim 14, wherein the presence of ATP released during extension of the primer by the DNA polymerase is compared to the level of release from a primer for a different allele at the SNP, and wherein the release is at least 50% higher if the nucleic acids in the sample comprise the specific allele in the primer.

25. A reaction mixture for use in a method of claim 1.

26. A kit comprising at least one ARN and optional dNTP reagents for use in a method of claim 1.

27. A method for synthesis of an ATP-releasing nucleotide (ARN) having a structure 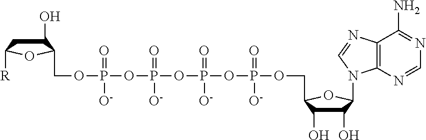
wherein R is where R is any purine or pyrimidine, or an analog thereof, the method comprising:
contacting salts of either (a) deoxynucleoside monophosphates (dNMPs) or (b) AMP with an activating agent; and
reacting the product of (a) with a salt of 5′-ATP or reacting the product of (b) with salts of a desired deoxynucleotide-5′-triphosphates (dNTP).

28. The method of claim 27, wherein the activating agent is carbonyldiimidazole or a carbodiimide agent.

29. The method of claim 27, wherein the salt is a tetra- or tri-alkylammonium salt.


SEQUENCE LISTING (29 sequences)

SEQ ID NO: 1 - DNA, 16 bp, H. sapiens
tcgagctagcggatga

SEQ ID NO: 2 - DNA, 36 bp, H. sapiens
gaggaaggaggaggaggaggtcatccgctagctcga

SEQ ID NO: 3 - DNA, 15 bp, H. sapiens
tctctcgtgcagact

SEQ ID NO: 4 - DNA, 20 bp, H. sapiens
gcaggtcgactctagaggat

SEQ ID NO: 5 - DNA, 50 bp, H. sapiens
tactacctcatcatttctctcgtgcagactcggactttaactatacaacc

SEQ ID NO: 6 - DNA, 36 bp, H. sapiens
tttttttttttttttttttttcatccgctagctcga

SEQ ID NO: 7 - DNA, 36 bp, H. sapiens
aaaaaaaaaaaaaaaaaaaatcatccgctagctcga

SEQ ID NO: 8 - DNA, 36 bp, H. sapiens
ggggggggggggggggggggtcatccgctagctcga

SEQ ID NO: 9 - DNA, 35 bp, H. sapiens
ccccccccccccccccccctcatccgctagctcga

SEQ ID NO: 10 - DNA, 16 bp, H. sapiens
tcgagctagcggatga

SEQ ID NO: 11 - DNA, 13 bp, H. sapiens
ctaggatcatagc

SEQ ID NO: 12 - DNA, 20 bp, H. sapiens
atggcgtgctatgatcctag

SEQ ID NO: 13 - DNA, 13 bp, H. sapiens
ctaggatcatagc

SEQ ID NO: 14 - DNA, 20 bp, H. sapiens
  misc_feature 7: n = A,T,C or G
atggcgngctatgatcctag

SEQ ID NO: 15 - DNA, 19 bp, H. sapiens
ggaaacagctatgaccatg

SEQ ID NO: 16 - DNA, 18 bp, H. sapiens
gtaaaacgacggccagtg

SEQ ID NO: 17 - DNA, 20 bp, H. sapiens
gcaggtcgactctagagctc

SEQ ID NO: 18 - DNA, 22 bp, H. sapiens
tgaggtagtaggttgtatagtt

SEQ ID NO: 19 - DNA, 21 bp, H. sapiens
tgaggtaggaggttgtatagt

SEQ ID NO: 20 - DNA, 21 bp, H. sapiens
tgaggtagtagtttgtacagt

SEQ ID NO: 21 - DNA, 21 bp, H. sapiens
tgaggtagtagtttgtgctgt

SEQ ID NO: 22 - RNA, 21 nt, H. sapiens
ugagguaguaguuugugcugu

SEQ ID NO: 23 - RNA, 22 nt, H. sapiens
ugagguaguagguuguauaugg

SEQ ID NO: 24 - DNA, 18 bp, H. sapiens
cactccatcgagatttca

SEQ ID NO: 25 - DNA, 18 bp, H. sapiens
cactccatcgagatttct

SEQ ID NO: 26 - DNA, 18 bp, H. sapiens
attctcgtctccacagac

SEQ ID NO: 27 - DNA, 18 bp, H. sapiens
attctcgtctccacagaa

SEQ ID NO: 28 - DNA, 18 bp, H. sapiens
cgtaggtcatgaactcag

SEQ ID NO: 29 - DNA, 18 bp, H. sapiens
cgtaggtcatgaactcaa